(19) United States
(12) Patent Application Publication
(10) Pub. No.: US 2020/0268253 A1
(43) Pub. Date: Aug. 27, 2020

(54) PHOTOACOUSTIC COMPUTED TOMOGRAPHY (PACT) SYSTEMS AND METHODS

(71) Applicants: California Institute of Technology, Pasadena, CA (US); Washington University, St. Louis, MO (US)

(72) Inventors: Lihong Wang, Arcadia, CA (US); Peng Hu, Pasadena, CA (US); Li Lin, Pasadena, CA (US)

(21) Appl. No.: 16/798,204

(22) Filed: Feb. 21, 2020

Related U.S. Application Data
(60) Provisional application No. 62/808,945, filed on Feb. 22, 2019.

(51) Int. Cl. A61B 5/00

(52) U.S. Cl. CPC A61B 5/0095; A61B 5/004

(57) ABSTRACT

Among the various aspects of the present disclosure is the provision of systems and methods of imaging using photoacoustic computed tomography.

### CROSS-REFERENCES TO RELATED APPLICATIONS

This application claims priority to and benefit of U.S. Provisional Patent Application No. 62/808,945, titled “PHOTOACOUSTIC COMPUTED TOMOGRAPHY” and filed on Feb. 22, 2019, which is hereby incorporated by reference in its entirety and for all purposes.

### FEDERALLY SPONSORED RESEARCH OR DEVELOPMENT

This invention was made with government support under Grant Nos. EB016963, EB016986, and CA186567 awarded by National Institutes of Health. The government has certain rights in the invention.

### FIELD

Certain embodiments generally relate to photoacoustic imaging, and more specifically, to methods and systems the employ photoacoustic computed tomography.

### BACKGROUND

Breast cancer is the second most common cancer to affect women in the United States and is the second ranked cause of cancer-related deaths. About 1 in 8 women in the United States will develop invasive breast cancer during their lifetime as discussed in Siegel, R. L., Miller, K. D. & Jemal, A., Cancer statistics, 2017, CA Cancer J. Clin. 67, pp. 7-30 (2017), which is hereby incorporated by reference for this discussion. Multiple large prospective clinical trials have demonstrated the importance of early detection in improving breast cancer survival as discussed, for example, in Dizon, D. S. et al., “Clinical cancer advances 2016: annual report on progress against cancer from the American Society of Clinical Oncology,” J. Clin. Oncol. 34, pp. 987-1011 (2016), Miller, A. B. et al., “Twenty five year follow-up for breast cancer incidence and mortality of the Canadian National Breast Screening Study: randomised screening trial,” BMJ 348, 366 (2014), and Burton, R. & Bell, R., “The global challenge of reducing breast cancer mortality,” Oncologist 18, pp. 1200-1201 (2013), which are hereby incorporated by reference in their entireties. While mammography is currently the gold standard used for breast cancer screening, it utilizes ionizing radiation and has lower sensitivity in women with dense breasts as discussed in Pinsky, R. W. & Helvie, M. A., “Mammographic breast density: effect on imaging and breast cancer risk,” J. Natl. Compr. Canc. Netw. 8, pp. 1157-1165 (2010) and Freer, P. E., Mammographic breast density: impact on breast cancer risk and implications for screening, Breast Imaging 35, e352140106 (2014), which are hereby incorporated by reference in their entireties for this discussion. Ultrasonography has been used as an adjunct to mammography, but can suffer from speckle artifacts and low specificity as discussed in Devolli-Disha, E., Manxhuka-Kerliu, S., Ymeri, H. & Kutllovci, A., “Comparative accuracy of mammography and ultrasound in women with breast symptoms according to age and breast density,” Bosn. J. Basic. Med. Sci. 9, pp. 131-136 (2009) and Hooley, R. J., Scoutt, L. M. & Philpotts, L. E., “Breast ultrasonography: state of the art,” Radiology 268, p. e13121606 (2013), which are hereby incorporated by reference in their entireties for this discussion. Magnetic resonance imaging (MRI) poses a large financial burden and requires the use of intravenous contrast agents that can cause allergy, kidney damage, and permanent deposition in the central nervous system, as discussed respectively in Murphy, K. J., Brunberg, J. A. & Cohan, R. H., “Adverse reactions to gadolinium contrast media: a review of 36 cases,”Am. J. Roentgenol. 167, pp. 847-849 (1996), Perazella, M. A., “Gadolinium-contrast toxicity in patients with kidney disease: nephrotoxicity and nephrogenic systemic fibrosis,” Curr. Drug Saf 3, pp. 67-75 (2008), and Ibrahim, D., Froberg, B., Wolf, A. & Rusyniak, D. E., “Heavy metal poisoning: clinical presentations and pathophysiology, Clin. Lab. Med. 26, pp. 67-97 (2006), which are hereby incorporated by reference in their entireties for this discussion. Diffuse optical tomography has been investigated to provide functional optical contrast. However, the spatial resolution of current prototypes may limit their clinical use as discussed in Choe, R. et al., “Diffuse optical tomography of breast cancer during neoadjuvant chemotherapy: a case study with comparison to MRI,” Med. Phys. 32,1128-1139 (2005) and Culver, J. P. et al., “Three-dimensional diffuse optical tomography in the parallel plane transmission geometry: evaluation of a hybrid frequency domain/continuous wave clinical system for breast imaging,”Med. Phys. 30, pp. 235-247 (2003), which are hereby incorporated by reference in their entireties for this discussion.

### SUMMARY

Certain aspects pertain to photoacoustic computed tomography (PACT) methods and/or systems that can be used, for example, to image breast tissue and other biological tissues.

Certain aspects pertain to photoacoustic computed tomography (PACT) systems. In one implementation, a PACT system comprises at least one pulsed or modulated light source, an ultrasonic transducer array comprising unfocused transducer elements, and a scanning mechanism configured to move and/or scan the ultrasonic transducer array along the axis. Each unfocused transducer element having a field-of-view in a range of 5 degrees to 30 degrees in a direction along an axis. In one example, the ultrasonic transducer array is a full-ring ultrasonic transducer array and the unfocused transducer elements are distributed around a circumference of a ring centered about the axis.

Certain aspects pertain to a photoacoustic computed tomography (PACT) methods. In one implementation, a PACT method comprises causing at least one pulsed light source to generate one or more light pulses configured to illuminate a specimen being imaged. The method further comprises controlling a scanning mechanism to move and/or scan the ultrasonic transducer array in a direction along an axis, wherein the ultrasonic transducer array includes a plurality of unfocused transducer elements, wherein the ultrasonic transducer array is moved/scanned in the direct along the axis while each of a plurality of unfocused transducer elements detects photoacoustic waves within a field-of-view in a range of 5 degrees to 30 degrees in the direction along the axis. In addition, the method comprises reconstructing a plurality of 2D images and/or a 3D volumetric image using photoacoustic signals recorded while the scanning mechanism moves/scans the ultrasonic transducer array in the direction along the axis.

Certain aspects pertain to a method of imaging breast issue of a subject. The method comprises providing breast tissue being imaged, scanning the breast tissue within the imaging field using photoacoustic computed tomography, and reconstructing a 3D volumetric image using 3D back projection and/or a plurality of 2D images using 2D back projection.

These and other features are described in more detail below with reference to the associated drawings.

### BRIEF DESCRIPTION OF THE DRAWINGS

FIG. 1 is a schematic diagram of components of a PACT system, according to certain implementations.

FIG. 2A is an illustration of a donut beam having a ring diameter of 6 cm, according to one aspect.

FIG. 2B is an illustration of simulated optical fluence in breast tissue at 2 cm depth when illuminated by the donut beam shown in FIG. 2A.

FIG. 2C is an illustration of a Gaussian-shaped beam with FWHM of approximately 6 cm, according to one aspect.

FIG. 2D is an illustration of simulated optical fluence in breast tissue at 2 cm depth when illuminated by the Gaussian-shaped beam shown in FIG. 2C.

FIG. 3A is an illustration of a simulated acoustic diffraction field in the elevational direction of two unfocused transducer elements of a full-ring ultrasonic transducer array, according to one implementation.

FIG. 3B is a plot of a line profile reconstructed by 2D back-projection and in the elevational direction of a carbon particle of 20-50 μm at the center of the ring of the full-ring ultrasonic transducer array with unfocused transducer elements in FIG. 3A.

FIG. 3C is a plot of a line profile reconstructed by 3D back-projection in the elevational direction of the same carbon particle at the center of the ring of the full-ring ultrasonic transducer array with unfocused transducer elements in FIG. 3A.

FIG. 4A is a graph of raw radio frequency (RF) signal from each unfocused ultrasonic transducer element corresponding to a point photoacoustic source at the center of the full-ring ultrasonic transducer array, according to an aspect.

FIG. 4B is a graph of the Fourier-transform amplitude of each RF signal in FIG. 4A.

FIG. 5A is a graph of a maximum amplitude projection (MAP) image of two crossed tungsten wires imaged by PACT system having a full-ring ultrasonic transducer array with unfocused elements, according to an aspect.

FIG. 5B is a graph of a photoacoustic amplitude distribution along the dashed line in FIG. 5A.

FIG. 6A is a perspective cut-away view of components of a PACT system, according to an implementation.

FIG. 6B is a perspective view of components of the PACT system partially shown in FIG. 6A, according to an implementation.

FIG. 7A is a perspective view of an example of a patient bed with a PACT system locating underneath, according to an implementation.

FIG. 7B is a perspective, close-up view of a portion of the patient bed shown in FIG. 7B.

FIG. 8 is a schematic signal flow diagram between components of a PACT system, according to an aspect.

FIG. 9 is a flowchart of PACT method, according to certain aspects.

FIG. 10 is a flowchart of operations of an exemplary mass detection method that performs elastographic evaluation on a plurality of 2D images, according to one aspect.

FIG. 11 is a flowchart of operations of an exemplary mass detection procedure that performs an automated mass segmentation process of a volumetric 3D image acquired in 3D mode, according to one aspect.

FIG. 12A is a flowchart of operations of a universal back-projection process that can be used to reconstruct either a 2D image or a 3D image, according to an aspect.

FIG. 12B is a flowchart of additional operations of the universal back-projection process in FIG. 12A as used for the 3D image, according to an implementation.

FIG. 13A is a PACT image at a depth of 0.5 cm from the nipple, according to an implementation.

FIG. 13B is a PACT image at a depth of 1.5 cm from the nipple, according to an implementation.

FIG. 13C is a PACT image at a depth of 2.5 cm from the nipple, according to an implementation.

FIG. 13D is a PACT image at a depth of 4.0 cm from the nipple, according to an implementation.

FIG. 14A is an image of the same specimen from FIG. 13A with color-encoded depths, according to an implementation.

FIG. 14B is a close-up view of the region outlined in FIG. 14A with two vessels, according to an implementation.

FIG. 14C is a graph of line spread plots of the two vessels identified in FIG. 14B, according to an implementation.

FIG. 15A is an illustration with a numerically-simulated image of a cylinder and an experimental image of a rubber cylinder, according to an implementation.

FIG. 15B is a plot of photoacoustic amplitude distributions along the normal directions of the dashed lines in FIG. 15A of the numerically-simulated cylinder and the rubber cylinder, according to an implementation.

FIG. 15C is a plot of correlation coefficients between numerical cylinders with different diameters and the rubber cylinder, according to an implementation.

FIG. 16A is an illustration with a numerically-simulated image of a cylinder with a diameter of 1.04 mm and an in vivo image of a section of a human blood vessel, according to an implementation.

FIG. 16B is a plot of photoacoustic amplitude distributions along the normal directions of the dashed lines in FIG. 16A of the numerically-simulated cylinder and the blood vessel, according to an implementation.

FIG. 16C is a plot of correlation coefficients between numerical cylinders with different diameters and the blood vessel, according to an implementation.

FIG. 17 is a PACT image of a healthy breast with the selected vessel tree in the breast with the five vessel bifurcations, according to an implementation.

FIG. 18 is a plot of the average junction exponents of the eight subjects, according to an implementation.

FIG. 19 is a heartbeat-encoded arterial network mapping of a breast cross-sectional image of a healthy breast from a PACT system, according to an implementation.

FIG. 20 is a plot of the pixel value fluctuation of the one artery and the one vein highlighted by dots in FIG. 19, according to an implementation.

FIG. 21 is a plot in the Fourier domain of the pixel value fluctuations in FIG. 20.

FIG. 22 is a plot of the noise-equivalent molar concentration (NEC) values plotted for arterial vessels with different diameters at different depths, according to an implementation.

FIG. 23A are images of a breast of the first patient P1, according to an aspect.

FIG. 23B are images of a breast of the second patient P2, according to an aspect.

FIG. 23C are images of a breast of the third patient P3, according to an aspect.

FIG. 23D are images of a breast of the fourth patient P4, according to an aspect.

FIG. 23E are images of a breast of the fifth patient P5, according to an aspect.

FIG. 23F are images of a breast of the sixth patient P6, according to an aspect.

FIG. 23G are images of a right breast of the seventh patient P7, according to an aspect.

FIG. 23H are images of a left breast of the seventh patient P7, according to an aspect.

FIG. 24A are images of a breast of the first patient P1, according to an aspect.

FIG. 24B are images of a breast of the second patient P2, according to an aspect.

FIG. 24C are images of a breast of the third patient P3, according to an aspect.

FIG. 24D are images of a breast of the fourth patient P4, according to an aspect.

FIG. 24E are images of a breast of the fifth patient P5, according to an aspect.

FIG. 24F are images of a breast of the sixth patient P6, according to an aspect.

FIG. 24G are images of a right breast of the seventh patient P7, according to an aspect.

FIG. 24H are images of a left breast of the seventh patient P7, according to an aspect.

FIG. 25A is a PACT image of a cross-sectional image of the phantom acquired by the PACT system, according to an implementation

FIG. 25B is a PACT elastographic image of the cross-section in FIG. 25A.

FIG. 26 is a plot of the receiver operating characteristic (ROC) curves of breast tumor detection based on blood vessel density, according to an aspect.

FIG. 27 is a bar chart of the average vessel density in each tumor and the surrounding normal breast tissue, according to an aspect.

FIG. 28 is a bar chart of the relative area change in each tumor and the surrounding normal breast tissue caused by breathing, according to an aspect.

FIG. 29 is a bar chart of the longest dimension and center depth of each tumor, according to an aspect.

FIG. 30 is a plot of the receiver operating characteristic (ROC) curve of tumor identification based on the sizes of the contiguous high vessel density regions, according to an implementation.

FIG. 31A an illustration of images of the left breast of the first patient P1, according to an aspect.

FIG. 31B an illustration of images of the breasts of the second patient P2, according to an aspect.

FIG. 31C an illustration of images of the breasts of the third patient P3, according to an aspect.

FIG. 31D an illustration of images of the breasts of the fourth patient P4, according to an aspect.

FIG. 31E an illustration of images of the breasts of the fifth patient P5, according to an aspect.

FIG. 31F an illustration of images of the breasts of the sixth patient P6, according to an aspect.

FIG. 31G an illustration of images of the breasts of the seventh patient P7, according to an aspect.

FIG. 32 is an illustration of three PACT images of breasts, according to an implementation.

FIG. 33 is a plot of the average vessel densities of tumors and surrounding normal tissues, according to an implementation.

FIG. 34 is a plot of the average vessel density ratio, according to an implementation.

FIG. 35 is a table of sensitivities and specificities of tumor detection based on vessel-density thresholds obtained from the training data sets, according to an implementation.

FIG. 36 is a PACT image of a cancerous breast, according to an implementation.

FIG. 37 is a plot of the relative area change over time for both the tumor and the normal tissue, according to an implementation.

These and other features are described in more detail below with reference to the associated drawings.

### DETAILED DESCRIPTION

Different aspects are described below with reference to the accompanying drawings. The features illustrated in the drawings may not be to scale. Different aspects are described below with reference to the accompanying drawings. The features illustrated in the drawings may not be to scale. In the following description, numerous specific details are set forth in order to provide a thorough understanding of the presented embodiments. The disclosed embodiments may be practiced without one or more of these specific details. In other instances, well-known operations have not been described in detail to avoid unnecessarily obscuring the disclosed embodiments. While the disclosed embodiments will be described in conjunction with the specific embodiments, it will be understood that it is not intended to limit the disclosed embodiments.

Certain aspects pertain to photoacoustic computed tomography (PACT) methods. In one aspect, a PACT method performs elastographic evaluation of a plurality of 2D photoacoustic images acquired at a high frame rate (e.g., at least 10 Hz) at a cross-sectional depth. In some cases, the elastographic evaluation is performed for each of a plurality of depths. The 2D photoacoustic images may be acquired by a PACT system or other imaging system that can acquires 2D images at a high frame rate. The high imaging speed allows for differentiation in compliance (or stiffness) between tumors and surrounding normal tissue. Tumors tend to be less compliant, deforming to a lesser extent, than surrounding normal tissue. This PACT method can differentiate between tumors and surrounding normal tissue by analyzing the differential compliance in the 2D images taken at high speed of a cross-section. This differential compliance may be used as another contrast for detecting masses of interest in biological tissues.

Certain aspects pertain to photoacoustic computed tomography (PACT) systems. In one aspect, a PACT system is configured to reconstruct a 3D volumetric image, e.g., to image detailed angiographic structures in human breasts and other biological tissues. For example, certain PACT systems can image with deep penetration depth (e.g., 4 cm in vivo) at high spatial resolution (e.g., 255-μm in-plane resolution) and/or high temporal resolutions (e.g., 10-Hz frame rate). These PACT systems and methods can be used to scan an ultrasonic transducer array through the depth of a breast within a single breath hold, which is typically less than about 15 seconds or less than about 10 seconds. A 3D back projection technique can be used to reconstruct a 3D volumetric with negligible breathing-induced motion artifacts from the photoacoustic data. Other examples of specimens that can be imaged using these PACT systems and methods would be contemplated.

In certain implementations, PACT techniques may be used to clearly image and reveal tumors by observing higher blood vessel densities associated with tumors at high spatial resolution. This imaging capability shows early promise for high sensitivity in radiographically-dense breasts. In addition to blood vessel imaging, high imaging speed-enabled dynamic implementations of certain PACT techniques, such as those that utilize photoacoustic elastography, may be able to identify tumors by showing less compliance in the tumors in comparison to surrounding tissue. Certain implementations of PACT techniques are capable of imaging breasts with sizes ranging from B cup to DD cup, and skin pigmentations ranging from light to dark. Certain PACT techniques can be used to identify tumors without any ionizing radiation or exogenous contrast, and thus, avoid the associated health risks.

In certain implementations, PACT techniques employ a single-breath-hold 3D imaging mode where the ultrasonic transducer array with unfocused elements is scanned through a depth of the breast (or other biological tissue) during the duration of a typical breath hold (about 15 sec). The unfocused transducer elements detect photoacoustic waves within their angle of view. The data acquired during this mode of operation can reveal detailed angiographic structures in human breasts. Certain SBH-PACT techniques feature penetration depth (e.g., up to 4 cm in vivo) with high spatial and/or temporal resolutions (e.g., with 255-μm in-plane resolution and/or a 10-Hz two-dimensional (2D) frame rate). By scanning the breast within a single breath hold, a volumetric image can be acquired and subsequently reconstructed utilizing 3D back projection with negligible breathing induced motion artifacts. By employing a single-breath-hold data acquisition mode, these PACT systems and methods may clearly reveal tumors by observing higher blood vessel densities associated with tumors at high spatial resolution, showing early promise for high sensitivity in radiographically dense breasts. Other examples of specimens that can be held or kept from moving during the time period of about 15 seconds and imaged with this technique would be contemplated.

In addition to blood vessel imaging, certain implementations of PACT techniques employ a dynamic 2D imaging mode where the ultrasonic transducer array with unfocused elements is moved to one or more depths (elevational locations) of the breast or other biological tissue. At each depth, the unfocused transducer elements detect photoacoustic signals from their angle of view. By employing a dynamic mode, these techniques may be used to identify tumors by showing less compliance in the tumors as compared to the surrounding tissue.

### I. Photoacoustic Computed Tomography (PACT) Introduction

Photoacoustic computed tomography (PACT) techniques ultrasonically image optical contrast via the photoacoustic effect. PACT techniques may be able to break through the about 1 mm optical diffusion limit on penetration for high-resolution optical imaging in deep tissues. Some examples of photoacoustic tomography are described in Xia, J., Yao, J. & Wang, L. V., “Photoacoustic tomography: principles and advances,” Electromagn. Waves 147, pp. 1-22 (2015) and Razansky, D. et al., “Multispectral opto-acoustic tomography of deep-seated fluorescent proteins in vivo,” Nat. Photon. 3, 412-417 (2009), which are hereby incorporated by reference in their entireties. PACT techniques combine the functional optical contrast of diffuse optical tomography and the high spatial resolution of ultrasonography. The rich contrast from optical absorption, which is related to various intrinsic and extrinsic contrast origins, enables PACT techniques to be able to perform structural, functional, and molecular imaging. A discussion of employing photoacoustic tomography for functional and molecular imaging can be found in Yao, J., Xia, J. & Wang, L. V., “Multi-scale functional and molecular photoacoustic tomography,” Ultrason. Imag. 38, pp. 44-62 (2016), which is hereby incorporated by reference in its entirety.

When a short-pulsed laser irradiates biological tissues, some of the delivered energy is absorbed and converted into heat, leading to transient thermoelastic expansion generating ultrasonic waves or emissions (sometimes referred to herein as “photoacoustic waves” or “PA waves”). The ultrasonic waves can be measured by an ultrasonic transducer to reconstruct the optical absorption distribution in the tissue to generate photoacoustic images as discussed in Zhou, Y., Yao, J. & Wang, L. V., “Tutorial on photoacoustic tomography,” J. Biomed. Opt. 21, 061007 (2016), which is hereby incorporated by reference in its entirety. For example, the 1/e attenuation coefficient for light in an him average breast is in a range of 1.0 to 1.3 cm ⁻¹ as discussed in Durduran, T., “Bulk optical properties of healthy female breast tissue,” Phys. Med. Biol. 47, pp. 2847-2861 (2002), which is hereby incorporated by reference in its entirety. Whereas the 1/e attenuation coefficient for mammographic X-rays is in a range of 0.5-0.8 cm⁻¹as discussed in Heine, J. J. & Thomas, J. A, “Effective X-ray attenuation coefficient measurements from two full field digital mammography systems for data calibration applications,” Biomed. Eng. Online 7, 13 (2008), which is hereby incorporated by reference in its entirety. That is, the optical absorption contrast of soft tissue is much higher than X-ray contrast as discussed in Fang, Q. et al., “Combined optical imaging and mammography of the healthy breast: optical contrast derived from breast structure and compression,” IEEE Trans. Med. Imag. 28, pp. 30-42 (2009), which is hereby incorporated by reference in its entirety. In some cases, PACT techniques may provide high spatial and temporal resolutions in imaging breast tissue with sufficiently deep nonionizing optical penetration. Some examples of photoacoustic imaging are discussed in Mallidi, S., Luke, G. P. & Emelianov, S., “Photoacoustic imaging in cancer detection, diagnosis, and treatment guidance,” Trends Biotechnol. 29, 213-221 (2011) and Wang, L. V., “Multiscale photoacoustic microscopy and computed tomography,” Nat. Photon. 3, 503-509 (2009), which are hereby incorporated by reference in their entireties. As the principal optical absorber in the near infrared region, hemoglobin provides an endogenous contrast for imaging of blood vessels.

Generally speaking, a high density of blood vessels tends to correlate with angiogenesis. A discussion of angiogenesis can be found in Weidner, N., Semple, J. P., Welch, W. R. & Folkman, J., “Tumor angiogenesis and metastasis—correlation in invasive breast carcinoma,” N. Engl. J. Med. 324, 1-8 (1991), Schneider, B. P. & Miller, K. D., “Angiogenesis of breast cancer,” J. Clin. Oncol. 23, 1782-1790 (2005), and Reynolds, A. R. et al., “Stimulation of tumor growth and angiogenesis by low concentrations of RGD-mimetic integrin inhibitors,” Nat. Med. 15, 392-400 (2009), which are hereby incorporated by reference in their entireties. Angiogenesis may play an important role in tumor growth and metastasis as discussed in Folkman, J., “Role of angiogenesis in tumor growth and metastasis,” Semin. Oncol. 29, 15-18 (2002), which is hereby incorporated by reference in its entirety.

Some photoacoustic imaging systems that have been used to image human breasts are mentioned in Toi, M. et al., “Visualization of tumor-related blood vessels in human breast by photoacoustic imaging system with a hemispherical detector array,” Sci. Rep. 7, 41970 (2017) (hereinafter referred to as “Toi”), Kruger, R. A. et al., “Dedicated 3D photoacoustic breast imaging,”Med. Phys. 40, 113301 (2013) (hereinafter referred to as “Kruger”) Wang, D. et al., “Deep tissue photoacoustic computed tomography with fast and compact laser system,” Biomed. Opt. Express 8, 112-123 (2017), Heijblom, M., Steenbergen, W. & Manohar, S., “Clinical photoacoustic breast imaging: the Twente experience,” IEEE Pulse 6, 42-46 (2015), Heijblom, M. et al., “Photoacoustic image patterns of breast carcinoma and comparisons with magnetic resonance imaging and vascular stained histopathology,” Sci. Rep. 5, 11778 (2015), Fakhrejahani, E. et al., “Clinical report on the first prototype of a photoacoustic tomography system with dual illumination for breast cancer imaging,” PloS ONE 10, e0142287 (2015), Kitai, T. et al., “Photoacoustic mammography: initial clinical results,” Breast Cancer 21, 146-153 (2014), Ermilov, S. A. et al., “Laser optoacoustic imaging system for detection of breast cancer,” J. Biomed. Opt. 14, 024007 (2009), Ke, H., Erpelding, T. N., Jankovic, L., Liu, C. & Wang, L. V., “Performance characterization of an integrated ultrasound, photoacoustic, and thermoacoustic imaging system,” J. Biomed. Opt. 17, 056010 (2012), Li, X., Heldermon, C. D., Yao, L., Xi, L. & Jiang, H., “High resolution functional photoacoustic tomography of breast cancer,”Med. Phys. 42, 5321-5328 (2015), which are hereby incorporated by reference in their entireties. These photoacoustic imaging systems may not meet the following requirements for breast imaging: (1) sufficient penetration depth to accommodate most breast sizes and skin colors, (2) high spatial resolution to reveal detailed angiographic structures, (3) high temporal resolution to minimize motion artifacts and enable dynamic or functional studies, (4) minimal limited-view artifacts, and (5) sufficient noise-equivalent sensitivity and contrast-to noise ratio to detect breast masses. Specifically, these photoacoustic imaging systems' have limitations mainly arising from their long scanning times and/or limited-view apertures (i.e., missing data or a<2π steradian solid angle).

For example, Toi and Kruger describe photoacoustic imaging systems that employ a hemispherical detector array and scan in a spiral pattern on a plane. Tumor detection with these systems was limited by respiratory motion artifacts resulting from long scanning time of about 4 minutes. Small tumor vessels, which often occur in small clusters were difficult to image with partial data and even more difficult to be coregistered with these systems. As anther example, others have planar transducer arrays and arc arrays for breast imaging. The limited views in these systems lowered their overall performance as discussed in Cox, B. T., Arridge, S. R. & Beard, P. C., “Photoacoustic tomography with a limited-aperture planar sensor and a reverberant cavity,” Inverse Probl. 23, S95-S112 (2007) and Huang, B., Xia, J., Maslov, K. & Wang, L. V., “Improving limited-view photoacoustic tomography with an acoustic reflector,” J. Biomed. Opt. 18, 110505 (2013), which are hereby incorporated by reference in their entireties. Consequently, most blood vessels were not well visualized in their images. The same problem occurred with linear transducer arrays, either fixed in position or scanned. One photoacoustic imaging system uses a ring-shaped array of 32 elements; however, however, the 32-element array generates a very limited field of view due to the sparse sampling. Accordingly, the system is not able to clearly reveal blood vessels in the breast as discussed in Li, L. et al., “Single-impulse panoramic photoacoustic computed tomography of small animal whole-body dynamics at high spatiotemporal resolution,” Nat. BME 1, 0071 (2017), which is hereby incorporated by reference in its entirety.

### II. Photoacoustic Computed Tomography (PACT) Systems

Certain aspects disclosed herein relate to PACT systems and methods. In certain implementations, PACT systems may be able to satisfy all the requirements for breast imaging discussed in Section I above. For example, in one implementation, a PACT system (i) combines 1064-nm light illumination and a 2.25-MHz unfocused ultrasonic transducer array to be able to achieve up to 4 cm in vivo imaging depth and a 255 μm in-plane resolution (approximately four times finer than that of contrast enhanced MRI. An example of MRI breast imaging is described in Lehman, C. D. & Schnall, M. D., “Imaging in breast cancer: magnetic resonance imaging,” Breast Cancer Res. 7, 215-219 (2005), which is hereby incorporated by reference in its entirety) to meet factors 1 and 2, (ii) is equipped with one-to-one mapped signal amplification and data acquisition (DAQ) circuits to be able to obtain an entire 2D cross-sectional breast image with a single laser pulse, or obtain a volumetric 3D image of the entire breast by fast elevational scanning within a single breath-hold (e.g., about 15 seconds) meeting factor 3, and (iii) has a 10 Hz 2D frame rate, currently limited by the laser repetition rate, allowing the system to observe biological dynamics in a cross-section associated with respiration and heartbeats without motion artifacts meeting factor 4, and (iv) includes a full-ring 512-element ultrasonic transducer array that enables the system to have a full-view fidelity in 2D imaging planes and delivers high image quality, meeting factor 5. Moreover, in certain implementations, a PACT system employs an illumination method and signal amplification that may be able to achieve sufficient noise-equivalent sensitivity that clearly reveal detailed angiographic structures both inside and outside breast tumors without the use of exogenous contrast agents.

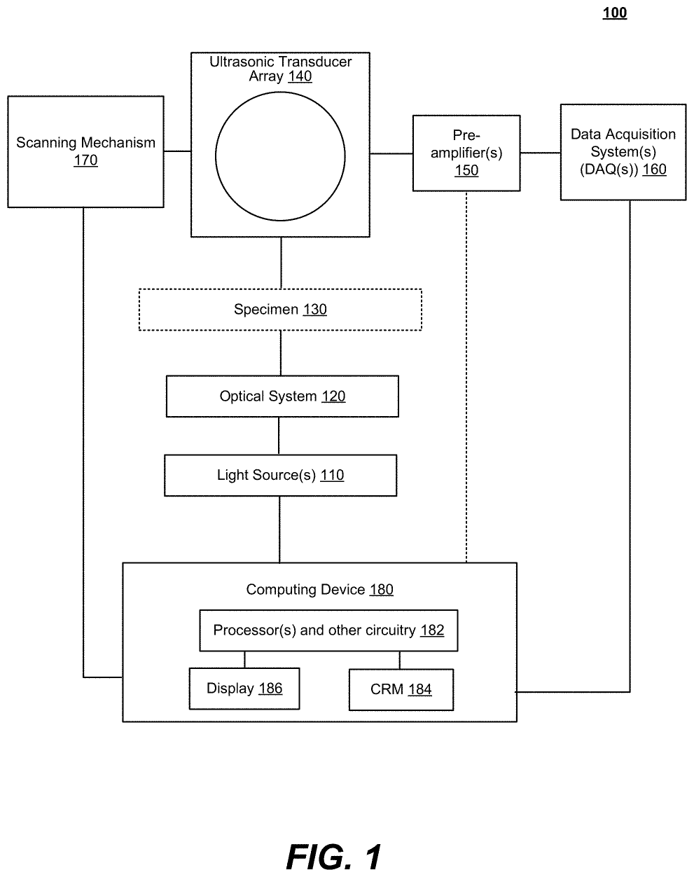

FIG. 1 is a schematic diagram of components of a PACT system 100, according to certain implementations. The PACT system 100 includes one or more light sources 110 (e.g., a pulsed laser) that can generate pulsed or modulated light, an optical system 120, and a specimen 130 being imaged during operation. The specimen 130 may be located in a specimen-receiving device for receiving and/or holding a specimen (e.g., a human breast) being imaged by the PACT system 100. The illustrated example shows the optical system 120 in optical communication with the light source(s) 110 to receive light during operation. The optical system 120 includes one or more optical components configured to propagate light to the specimen-receiving device to illuminate the specimen 130. In some cases, the optical system 120 is also configured to convert a light beam into shaped illumination such as donut-shaped illumination (sometimes referred to herein as “donut illumination” or a “donut beam”) as might be used, for example, to illuminate a human breast. The specimen 130 is in optical communication with the optical system 120 to receive illumination, such as, e.g., the donut beam, to illuminate the specimen 130 being imaged during operation. In another aspect, a uniform circular illumination can be used. A beam of circular illumination can be generated by employing an engineered diffuser such as, e.g., an EDC 15 diffuser made by RPC Photonics®.

The PACT system 100 also includes an ultrasonic transducer array 140 that can be coupled to or otherwise in acoustic communication with the specimen 130 to receive photoacoustic signals induced by the illumination. The PACT system 100 also includes one or more preamplifiers 150 and one or more data acquisition systems (DAQs) 160. The one or more pre-amplifiers 150 are in electrical communication with the ultrasonic transducer array 140 to receive a signal or signals. The DAQ(s) are in electrical communication with the pre-amplifier(s) 150 to receive a signal or signals. The PACT system 100 also includes a scanning mechanism 170 coupled to or otherwise operably connected to the ultrasonic transducer array 140, e.g., to move the ultrasonic transducer array 140 to one or more elevational positions and/or scan the ultrasonic transducer array 140 between two elevational positions. The PACT system 100 also includes a computing device 180 having one or more processors or other circuitry 182, a display 186 in electrical communication with the processor(s) 182, and a computer readable medium (CRM) 184 in electronic communication with the processor(s) 182. The computing device 180 is also in electronic communication with the light source(s) 110 to send control signals. The computing device 180 is in electrical communication with the DAQ(s) 160 to receive data transmissions and/or to send control signal(s). The computing device 180 is in electrical communication with the (DAQs) 160 to receive data transmissions. Optionally (denoted by dashed line), the computing device 180 is also in electronic communication with the one or more pre-amplifiers 150 to send control signal(s), e.g., to adjust the amplification. The electrical communication between system components of the PACT system 100 may be in wired and/or wireless form. The electrical communications may be able to provide power in addition to communicate signals in some cases.

In certain aspects, a PACT system includes a light source (e.g., a pulsed laser) that can generate pulsed or modulated illumination. In some cases, the light source is configured to generate pulsed or modulated light at a near-infrared wavelength or a narrow band of near-infrared wavelengths. For example, the light source may be a pulsed laser that can generate near infrared pulses having a wavelength or narrow band of wavelengths in a range from about 700 nm to about 1000 nm. As another example, the light source may be a pulsed laser that can generate near infrared pulses having a wavelength or narrow band of wavelengths in a range from about 600 nm to about 1100 nm. In yet another example, the light source may be a pulsed laser that can generate near infrared pulses with a wavelength or narrow band of wavelengths greater than 760 nm. In yet another example, the light source may be a pulsed laser that can generate near infrared pulses with a wavelength or narrow band of wavelengths greater than 1000 nm. In one implementation, the light source is a pulsed laser that can generate a 1064-nm laser beam. A commercially-available example of such as pulsed laser is the PRO-350-10, Quanta-Ray® laser with a 10-Hz pulse repetition rate and 8 ns-12 ns pulse width sold by Spectra-Physics®. The low optical attenuation of 1064 nm light or other near infrared light can be used to deeply penetrate (e.g., to a depth of 4 cm) biological tissues such as breast tissue. Imaging of biological tissues using near infrared light is discussed in Smith, A. M., Mancini, M. C. & Nie, S., “Bioimaging: second window for in vivo imaging,” Nat. Nanotechnol. 4, 710-711 (2009), which is hereby incorporated by reference in its entirety. Alternatively, the light source may be a continuous wave (CW) laser source that is chopped, modulated and/or gated to generate the pulsed or modulated illumination.

In implementations that have a light source in the form of a pulsed laser, the pulse repetition rate may be about 10-Hz in some cases, about 20-Hz in other cases, about 50-Hz in other cases, and about 100-Hz in other cases. In another aspect, the pulse repetition rate is in a range from about 10-Hz to about 100-Hz.

In one aspect, a light source of the PACT system is a tunable narrow-band pulsed laser such as, e.g., one of a quantum cascade laser, an interband cascade laser, an optical parametric oscillator, or other pulsed laser that can tuned to different narrow bands (e.g., near-infrared narrow bands of wavelengths). In other cases, the light source is a pulsed laser of a single wavelength or approximately a single wavelength.

In one aspect, the light source could be a combination of multiple same lasers. For example, multiple same lasers with a lower power for each of them. In another aspect, the light source could be a combination of multiple different lasers. For example, an optical parametric oscillator combined with an Nd:YAG laser.

An optical system of a PACT system includes one or more optical components (e.g., lens(es), optical filter(s), mirror(s), beam steering device(s), beam-splitter(s), optical fiber(s), relay(s), and/or beam combiner(s)) configured to propagate and/or alter light from a light source(s) to provide illumination to a specimen being imaged during operation. For example, the optical system may be configured to convert a light beam into shaped illumination such a donut beam that may be used, e.g., to circumferentially illuminate a human breast.

In one implementation, an optical system of a PACT system includes an axicon lens (e.g., an axicon lens having 25 mm diameter and a 160° apex angle) followed by an engineered diffuser (e.g., EDC-10-A-2s made by RPC Photonics) to convert a light beam into a donut beam. For example, the axicon lens may be positioned to receive a laser beam propagated from a pulsed laser source. The axicon lens can convert a single beam into a ring having a thickness and diameter and the engineered diffuser expands the ring into a donut beam. The donut beam may provide mass energy in homogenized, uniform illumination in deep tissue. An example donut-shaped illumination can be found in U.S. patent application Ser. No. 16/464,958, titled “SINGLE-IMPULSE PANORAMIC PHOTOACOUSTIC COMPUTED TOMOGRAPHY (SIP-PACT),” and filed on Nov. 29, 2017, which is hereby incorporated by reference in its entirety.

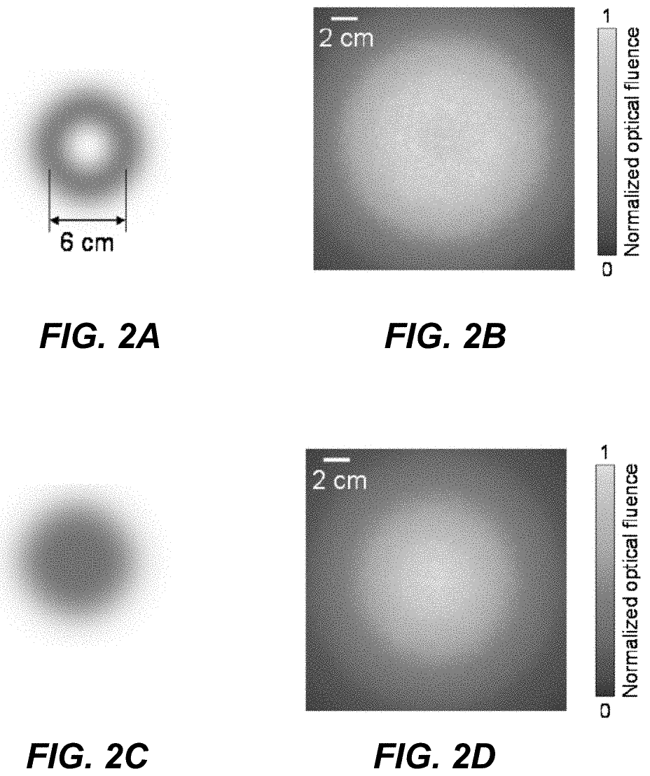

FIG. 2A is an illustration of a donut beam having a ring diameter of 6 cm, according to one aspect. FIG. 2B is an illustration, based on Monte Carlo simulation, of optical fluence in breast tissue at 2 cm depth that is illuminated by the donut beam shown in FIG. 2A. FIG. 2C is an illustration of a Gaussian-shaped beam with FWHM of approximately 6 cm, according to one aspect. FIG. 2D is an illustration, based on Monte Carlo simulation, of optical fluence in breast tissue at 2 cm depth when illuminated by the Gaussian-shaped beam shown in FIG. 2C. The optical fluence distributions in FIGS. 2B and 2D were based on a test set up that mimicked a compressed breast. To mimic a breast compressed against the chest wall, a cylindrical breast model was built with a height of 4 cm and a diameter of 15 cm. In the numerical model, the absorption coefficient (0.05 cm−1) and the reduced scattering coefficient (7 cm−1) inside the mimicked breast were selected for a 1064 nm wavelength.

Compared to a Gaussian beam, a donut beam may be able to provide more uniform illumination inside a breast and also deposit less energy on a nipple and areola, which have a higher concentration of pigment. The illumination wavelength of 1064 nm is characterized by low optical attenuation within breast tissue, which can enable sufficient optical penetration in breast tissue for PACT imaging. A discussion of optical properties of biological tissues can be found in Jacques, S. L., “Optical properties of biological tissues: a review,” Phys. Med. Biol. 58, 5007-5008 (2013), which is hereby incorporated by reference in its entirety.

When evaluating one implementation of a PACT system with an axicon lens having a 25 mm diameter and 160° apex angle followed by an engineered diffuser (e.g., EDC-10-A-2s made by RPC Photonics), a laser beam was broadened into a donut shape with an outer diameter of about 10 cm, depositing light with an average laser fluence of about 20 mJ/cm² on the breast surface. A laser fluence of 20 mJ/cm² is about ⅕ of the safety limit for laser exposure as provided by the American National Standards Institute in its American national standard for the safe use of lasers ANSI z136.1-2007, Laser Institute of America, Orlando, Fla. (2007), which is hereby incorporated by reference in its entirety. This outer radius will cover many breasts and provides adequate SNR in breast images. Another implementation with a more energetic laser could enlarge the illumination area and increase the optical fluence to potentially improve sensitivity further in mass detection. The sensitivity of photoacoustic microscopy is discussed in Yao, J. & Wang, L. V., “Sensitivity of photoacoustic microscopy,” Photoacoustics 2, 87-101 (2014), which is hereby incorporated by reference in its entirety.

The ultrasonic transducer array (e.g., ultrasonic transducer array 140 in FIG. 1) is coupled to or otherwise in acoustic communication with the specimen being imaged. In some cases, an acoustic medium such as an acoustic gel, water, or other medium capable of conveying ultrasound pulses, is provided at least partially between the specimen and the ultrasonic transducer array. In other cases, the acoustic medium may be omitted. The ultrasonic transducer array is acoustically coupled to the specimen to be able to detect photoacoustic waves induced by illumination and sample photoacoustic signals. These photoacoustic signals are indicative of the optical absorption of the specimen by the illumination. The ultrasonic transducer array includes a plurality of transducers (sometimes referred to herein as “transducer elements”) operable to collect multiple photoacoustic signals in parallel. Each transducer element in the array has an aperture (e.g., a flat-rectangular aperture) with a height and a width or pitch. The width or pitch may be about 1.35 mm in one aspect. The width or pitch may be in a range of 1.20 mm to 1.50 mm in another aspect. The height may be about 5 mm in one aspect. The height may be in a range of 2 mm to 10 mm in another aspect. By way of non-limiting example, using Eqn. 1 below, the diameter of the full-ring transducer array may be selected to satisfy a Nyquist spatial sampling criterion so that the transducer elements can sample photoacoustic signals with uniform or nearly uniform resolution within a field-of-view. For example, where N=512 and λ=500 μm, the diameter of the field-of-view may be selected to be 40.8 mm using Eqn. 1.

$$\frac{N λ}{2} = π D \\ Where \text{:} \\ N is the number of transducer elements , \\ λ is the wavelength corresponding to high \text{-} cut \text{-} off frequency of transducer elements \\ D is the diameter of the field \text{-} of \text{-} view \qquad ( Eqn . 1 )$$

In certain implementations, a full-ring ultrasonic transducer array is employed, e.g., to be able to provide 2D panoramic acoustic detection. The full-ring ultrasonic transducer array includes N transducer elements (e.g., 512-element full-ring ultrasonic transducer) distributed along the circumference of a ring having a diameter and an inter-element spacing. The ring diameter may be at least 220 mm in one aspect, may be at least 200 mm in one aspect, or may be at least 250 mm in one aspect. In one aspect, the ring diameter is in a range of about 150 mm to about 400 mm. The inter-element spacing may be less than or equal to about 1.0 mm in one aspect, less than or equal to 0.7 mm in one aspect, less than or equal to 1.5 mm in one aspect, or less than or equal to 2.0 mm in one aspect. In one aspect, the inter-element spacing is in a range of 0 mm to about 5 mm.

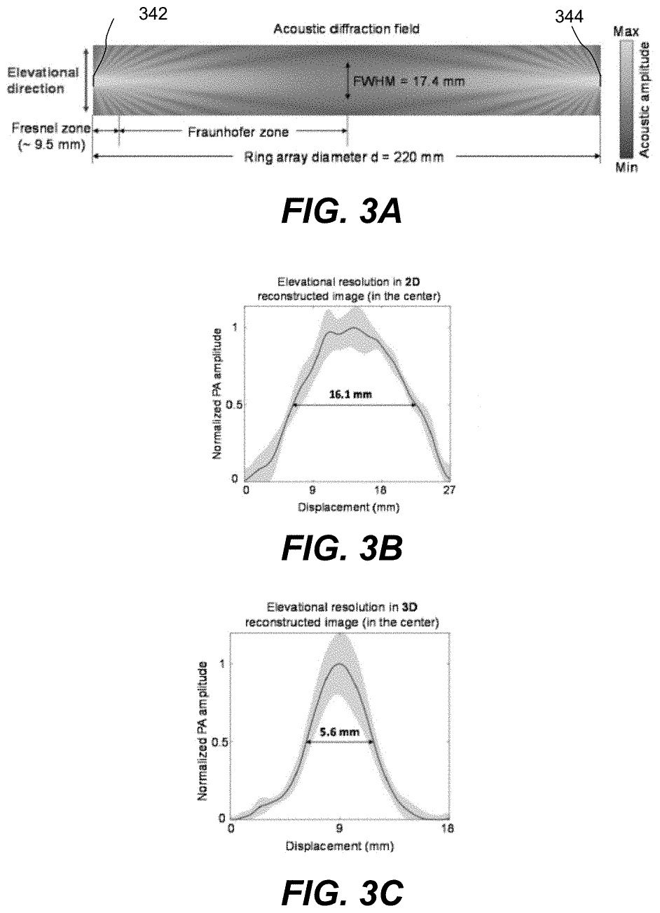

In one aspect, a full-ring ultrasonic transducer array with a ring of unfocused transducer elements is employed to sample both photoacoustic data at each laser pulse. An unfocused transducer element has a flared diffraction pattern with a diffraction angle of about 10 degrees as shown in FIG. 3A. Unfocused transducer elements having certain diffraction angles can provide elevational resolution in both 2D and 3D reconstruction (e.g., elevational resolution of 16.1 mm in 2D and 5.6 mm in 3D as shown in FIGS. 3B and 3C). In one aspect, a full-ring ultrasonic transducer array has unfocused transducer elements, each having a diffraction angle in a range of 5 degrees to 30 degrees. In another aspect, a full-ring ultrasonic transducer array has unfocused transducer elements, each having a diffraction angle of about 20 degrees. In yet another aspect, a full-ring ultrasonic transducer array has unfocused transducer elements, each having a diffraction angle in a range of 5 degrees to 30 degrees. In one aspect, each of the unfocused transducer elements has a central frequency in a range of 0.50 MHz to 2.25 MHz and a one-way bandwidth of more than 50%. In another aspect, each of the unfocused transducer elements has a central frequency in a range of 2.25 MHz to 10 MHz and a one-way bandwidth of more than 50%.

FIG. 3A is an illustration of a simulated acoustic diffraction field in the elevational direction of two unfocused transducer elements 342, 344 opposing each other in a full-ring ultrasonic transducer array having a ring diameter of 220 mm, according to one implementation. As shown, the height of each unfocused transducer element yields a divergence angle or diffraction angle in the elevational direction of about 9 degrees full width at half maximum (FWHM), yielding a flared diffraction pattern.

FIG. 3B is a line profile in the elevational direction of a carbon particle of 20-50 μm, placed at the center of the ring of the full-ring ultrasonic transducer array in FIG. 3A. The line profile in FIG. 3B is reconstructed by 2D back-projection of a universal back-projection (UBP) algorithm discussed in Section III. FIG. 3B shows that the elevational resolution of the 2D reconstructed image is 16.1 mm. FIG. 3C is a line profile in the elevational direction of the same carbon particle at the center of the ring of the full-ring ultrasonic transducer array in FIG. 3A. In this case, the line profile is reconstructed by a 3D back-projection discussed in Section III. FIG. 3C shows that the elevational resolution of the 3D reconstructed image is 5.6 mm. FIG. 3C shows that the 3D back projection algorithm can be used to reconstruct a volumetric image with an elevational resolution of 5.6 mm, which is about 3 times finer than that given by the 2D reconstruction algorithm. FIGS. 3B and 3C show that the flared diffraction pattern of these unfocused transducer elements 342, 344 provide adequate elevational resolution in both 2D and 3D reconstruction (e.g., finer than 20 mm in 2D and 10 mm in 3D)

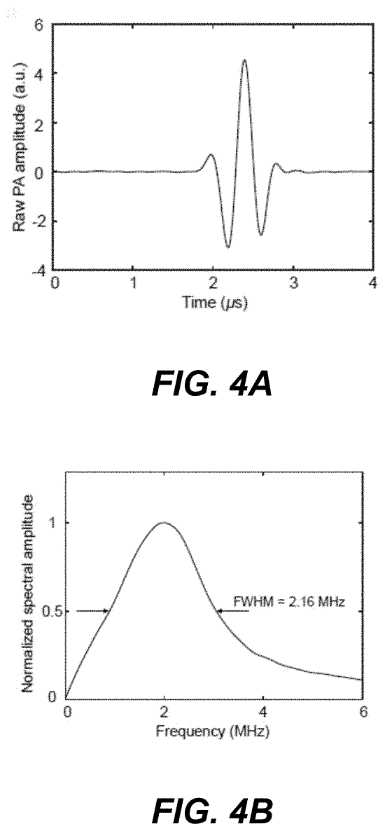

FIGS. 4A and 4B show the electrical impulse response of a full-ring ultrasonic transducer array having unfocused elements in a ring with a diameter of 220 mm, according to an aspect. The unfocused transducer elements have a central frequency of 2.25 MHz and a one-way bandwidth of more than 95%. FIG. 4A is a graph of raw radio frequency (RF) signal from each unfocused ultrasonic transducer element corresponding to a point photoacoustic source at the center of the full-ring ultrasonic transducer array, according to an implementation. The black solid line represents the standard deviation across the unfocused ultrasonic transducer elements. FIG. 4B is a graph of the Fourier-transform amplitude of each RF signal in FIG. 4A. FIG. 4B shows that the bandwidth of the transducer array is about 2.16 MHz. The black solid line represents the mean value of the spectral amplitude of all RF signals. The gray region represents the standard deviation across the unfocused ultrasonic transducer elements. The point source was created by fixing a carbon particle (e.g., 30-50 μm) in an agar phantom. The particle was small enough to be regarded as a spatial point source.

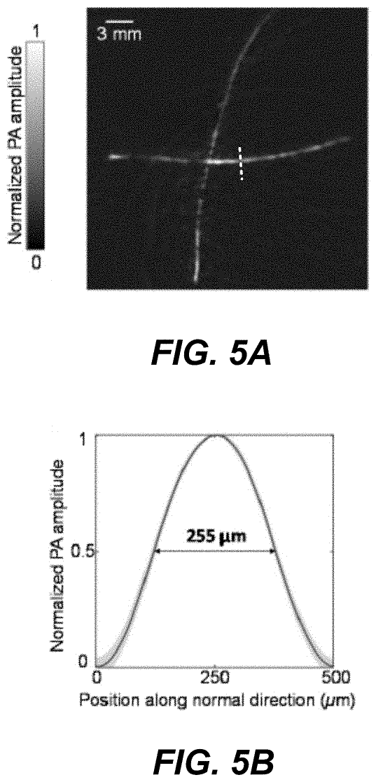

FIGS. 5A and 5B show a quantification of the in-plane resolution of the PACT system having a full-ring ultrasonic transducer array having unfocused elements in a ring with a diameter of 220 mm, the unfocused elements having a central frequency of 2.25 MHz and a one-way bandwidth of more than 95%, according to an aspect. FIG. 5A is a graph of a maximum amplitude projection (MAP) image of two crossed tungsten wires, each with a nominal diameter, of 13 μm. FIG. 5B is a graph of a photoacoustic amplitude distribution along the dashed line in FIG. 5A. As shown, the experimentally quantified in-plane resolution, defined as the FWHM of the amplitude distribution, was found to be 255 μm.

In certain aspects, donut-shaped optical illumination and panoramic acoustic detection are employed. The donut-shaped optical illumination and panoramic acoustic detection may provide uniform fluence distribution in deep tissue and in-plane coverage of ultrasound reception, respectively, delivering high image quality. Furthermore, considering the low cancer detection rate in mammography examinations (e.g., 0.41%), even though modern mammography uses a low dose of ionizing radiation, the risk-to-benefit ratio (e.g., 8%-17% for 40-50 year-old women) is considered high. The low cancer detection rate in mammography examinations is discussed in “Cancer Rate (per 1,000 examinations) and Cancer Detection Rate (per 1,000 examinations) for 1,838,372 Screening Mammography Examinations from 2004 to 2008 by Age —based on BCSC data through 2009,” NCI-funded Breast Cancer Surveillance Consortium (HHSN 261201100031C), which is hereby incorporated by reference. The risk-to-benefit ratios are discussed in Hendrick, R. E. & Tredennick, T., “Benefit to radiation risk of breast-specific gamma imaging compared with mammography in screening asymptomatic women with dense breasts,” Radiology 281, pp. 583-588 (2016) and Jung, H., “Assessment of usefulness and risk of mammography screening with exclusive attention to radiation risk,” Radiologe 41, 385-395 (2001), which are hereby incorporated by reference in their entirety. In comparison, the PACT system requires neither ionizing radiation nor an exogenous contrast agent, yielding zero risk.

In certain implementations, a PACT system includes a tank at least partially filed with acoustic medium such as a water tank (e.g., an acrylic water tank). The specimen being imaged may be located directly in the acoustic medium or in a portion of the tank that is submerged or otherwise located in the acoustic medium.

In certain implementations, a PACT system includes a specimen-receiving device for receiving and/or holding a specimen in place during the data acquisition phase. In one aspect, the specimen-receiving device includes a table or a patient bed and other components of the PACT system are located underneath the bed/table. In one aspect, the specimen-receiving device includes a housing for the ultrasonic transducer array where the ultrasonic transducer array is mounted on a stainless-steel rod (e.g., a rod having a 25 mm diameter) and is enclosed in a water tank.

Returning to FIG. 1, the PACT system 100 also includes a scanning mechanism 170 coupled to the ultrasonic transducer array 140 to be able to move and/or scan the ultrasonic transducer array 140 during operation, for example, along an axis in one or both directions. In one case, the scanning mechanism 170 can scan the ultrasonic transducer array 140 between two different positions along an axis (e.g., between z₁ and z₂ along a z-axis). In addition or alternatively, the scanning mechanism 170 can move the ultrasonic transducer array 140 to one or more positions (depths) along an axis (e.g., z₁, z₂, z₃, and z₄ along a z-axis) and hold at each position for a time period. In one aspect, the positions are uniformly separated by a certain distance such as, for example, about 1 cm, about 2 cm, about 3 cm. In one aspect, the distance may be defined by the elevational resolution of the 2d reconstructed image for the PACT implementation being used. For example, a distance of less than 16.1 mm may be used. The breast is usually compressed to a thickness of 3 cm to 4 cm. The 2d imaging mode acquire 2d images by collecting signals from a slice of tissue with a thickness of 16.1 mm. Therefore, a step size (distance between each elevation) of 1-2 cm is selected to cover the 3-4-cm thick breast by stopping at 2-4 elevational positions (monitoring the breath-induced motion for 5-60 seconds at each elevational position). The scanning mechanism 170 may include one or more mechanical motors to move the ultrasonic transducer array 140. The scanning mechanism may be, for example, a linear actuator, a linear ball screw assembly, a linear stage or one or more motorized scanning stages, etc.

The PACT system 100 also includes one or more pre-amplifiers 150 and one or more data acquisition systems (DAQ) 160. The pre-amplifier(s) 150 is in electrical communication with the ultrasonic transducer array 140 to be able to receive photoacoustic signals. The pre-amplifier(s) 150 can boost the photoacoustic signals received from the ultrasonic transducer array 140. The DAQ(s) 160 is in electrical communication with the pre-amplifier(s) 150 to be able to receive photoacoustic signals. The DAQ(s) 160 can process the photoacoustic signals, for example, digitize the signals and/or record the photoacoustic signals. In certain aspects, the DAQ(s) 160 include at least one digitizer.

According to certain implementations, a PACT system acquires images at a high imaging speed or frame rate. The high imaging speed helps avoid respiration-induced motion artifacts when scanning the ultrasonic transducer array between elevational positions in a single breath hold data acquisition mode. The high imaging speed may also help enable detection of breast tumors by detailing tumor-associated angiogenesis in a single elevation data acquisition mode. The frame rate may be about 10-Hz in some cases, about 50-Hz in other cases, and about 30-Hz in other cases. In another example, the frame rate is in a range from about 10-Hz to about 20-Hz. In another example, the frame rate is in a range from about 20-Hz to about 100-Hz.

In certain implementations, a PACT system includes a set of one or more DAQ devices and a set of one or more pre-amplifiers that together provide one-to-one mapped associations with the number of transducers in the ultrasonic transducer array. These one-to-one mapped associations allow for fully parallelized data acquisition of all ultrasonic transducer channels and avoids the need for multiplexing after each laser pulse excitation. With one-to-one mapped associations between pre-amplifiers and transducer elements, each ultrasound transducer element in the array is in electrical communication with one dedicated pre-amplifier channel (also referred to as “preamp channel”). The one dedicated pre-amplifier channel is configured to amplify only photoacoustic signals detected by the one associated/mapped ultrasound transducer. These one-to-one mapped associations between the transducers and the pre-amplifier channels allow for parallelized pre-amplification of the photoacoustic signals detected by the plurality of transducers in the ultrasound transducer array. With one-to-one mapped analog-to-digital sampling, each pre-amplifier is operatively coupled to a corresponding dedicated data channel of an analog-to-digital sampling device in a DAQ to enable parallelized analog-to-digital sampling of the plurality of pre-amplified PA signals. The pre-amplified PA signals produced by each individual preamp channel are received by a single dedicated data channel of the at least one analog-to-digital sampling devices. Any suitable number of pre-amplifier devices and/or DAQ devices may be used to provide the one-to-one mapping. For example, a PACT system may include four 128-channel DAQs (e.g., SonixDAQ made by Ultrasonix Medical ULC with 40 MHz sampling rate, 12-bit dynamic range, and programmable amplification up to 51 dB) in communication with four 128-channel pre-amplifiers to provide simultaneous one-to-one mapped associations with a 512-element transducer array. This PACT system can acquire photoacoustic signals from a cross section within 100 μs without multiplexing after each laser pulse excitation. The plurality of pre-amplifier channels may be directly coupled to the corresponding plurality of ultrasound transducers or may be coupled with electrical connecting cables. In one aspect, wireless communication may be employed.

In certain aspects, the pre-amplifier gain of the pre-amplifier channels is selected based on factors such as, for example, signal-to-noise ratio, operating parameters of other data acquisition and processing system components such as analog-to-digital sampling devices (digitizers) of the DAQs, signal amplifiers, buffers, and computing devices. In one aspect, the pre-amplifier gain is in a range that is high enough to enable transmission of the photoacoustic signals with minimal signal contamination, but below a gain that may saturate the dynamic ranges of the data acquisition (DAQ) system used to digitize the photoacoustic signals amplified by the pre-amplifier(s). In certain aspects, the gain of the plurality of pre-amplifier channels may be at least about 5 dB, at least about 7 dB, at least about 9 dB, at least about 11 dB, at least about 13 dB, at least about 15 dB, at least about 17 dB, at least about 19 dB, at least about 21 dB, at least about 23 dB, at least about 25 dB, or at least about 30 dB.

Returning to FIG. 1, the PACT system 100 also includes a computing device 180 having one or more processors or other circuitry 182, a display 186 in electrical communication with the processor(s) 182, and a computer readable medium (CRM) 184 in electronic communication with the processor(s) 182. The computing device 180 may be, for example, a personal computer, an embedded computer, a single board computer (e.g. Raspberry Pi or similar), a portable computation device (e.g. tablet), a controller, or any other computation device or system of devices capable of performing the functions described herein. The computing device 180 is in electronic communication with the scanning mechanism 170 to send control signals to control the movement and/or hold positions of the ultrasonic transducer array 140. The computing device 180 is also in electronic communication with the data acquisition unit(s) 160 to receive data transmissions with the photoacoustic signals and/or send control signals. The computing device 180 is also in electronic communication with the light source(s) 110 to send trigger signals to activate the light source(d), e.g., to send laser pulses. Optionally (denoted by dashed line), the computing device 180 is also in electronic communication with the one or more pre-amplifiers 150 to send control signals, e.g., to adjust the amplification. The processor(s) 182 are in electrical communication with the CRM 184 to store and/or retrieve data such as the photoacoustic signal data. The processor(s) 182 are in electrical communication with the user display 186 to receive input from a system operator and/or to send display data for displaying output.

The processor(s) 182 executes instructions stored on the CRM 184 to perform one or more operations of the PACT system 100. In certain implementations, the processor(s) 182 and/or one or more external processors execute instructions to perform one or more of 1) determining and communicating control signals to system components, 2) performing reconstruction algorithm(s) reconstructing a 2D image and/or a 3D image of the specimen using photoacoustic signal data; and 3) performing techniques (e.g., tumor segmentation and elastographic technique) that can identify tumors using the 2D and/or 3D PACT images. For example, the processor(s) 182 and/or one or more external processors may execute instructions that communicate control signals to the scanning mechanism 170 to scan the ultrasonic transducer array 140 along a z-axis between to two elevations (3D mode) or move the ultrasonic transducer array 140 to one or more different elevations (2D mode) and send control signals to the digitizer in the DAQ(s) 160 to simultaneously record photoacoustic signals received by ultrasonic transducer array 140 from the illuminated regions of the specimen.

In some implementations, the PACT system includes one or more communication interfaces (e.g., a universal serial bus (USB) interface). Communication interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that the various system components can be electrically coupled to communicate with various components over one or more of a variety of suitable interfaces and cables such as, for example, USB interfaces and cables, ribbon cables, Ethernet cables, among other suitable interfaces and cables.

In one aspect, the digitized radio frequency data from one or more DAQs (e.g., DAQs 160 in FIG. 1) is first stored in an onboard buffer, and then transferred to the computing device (e.g., computing device 180) through a universal serial bus 2.0. The DAQs may be configured to record PA signals within 100 μs after each laser pulse excitation. As another example, the digitized radio frequency data from one or more DAQs that do have an onboard buffer is transferred to the computer device through a universal serial bus 3.0. The DAQs may be configured to record PA signals within 200 μs after each laser pulse excitation.

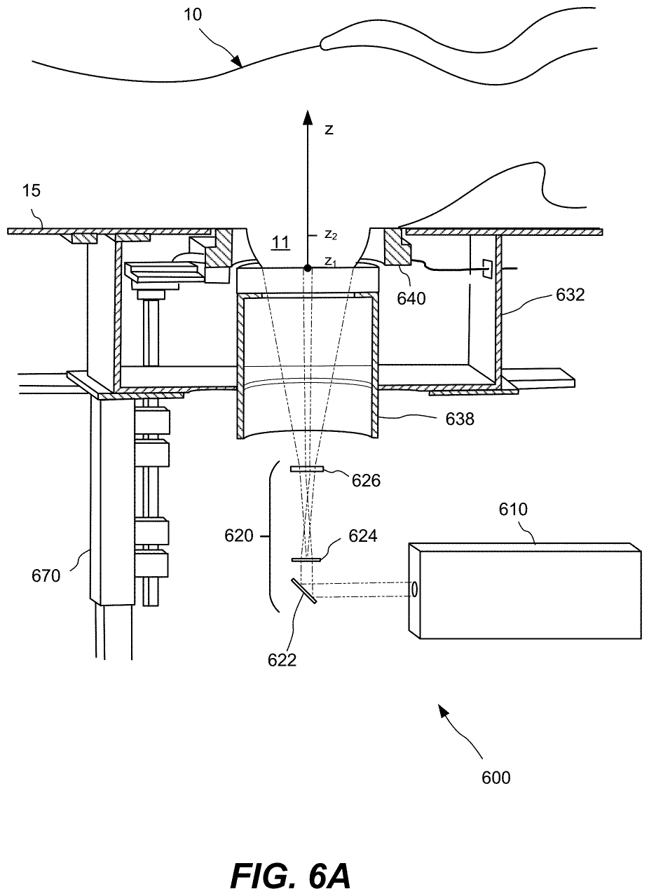

FIG. 6A is a perspective cut-away view of components of a PACT system 600 that can be implemented for breast imaging. In this illustration, the PACT system 600 is shown without pre-amplification system components, data acquisition system components, and a computing system. The PACT system 600 includes a light source 610 in the form of a pulsed 1064 nm laser source and an optical system 620 in optical communication with the pulsed laser 610 to receive laser pulses when it receives trigger signals during operation. The optical system 620 also includes a z-axis. The optical system 620 also includes a mirror 622 in optical communication with the pulsed laser 610 to receive light pulses, and an axicon lens 624 and an engineered diffuser 526 configured to convert light pulses into a donut beam. The PACT system 600 also includes a tank 532 with an acoustic medium such as water. The includes a cylinder 638 to support and compress the breast. The PACT system 600 also includes a 512-element ultrasonic transducer array 640 and a linear scanner 670 coupled to the ultrasonic transducer array 640 to be able to move the ultrasonic transducer array 640 to one or more elevational positions and/or scan the ultrasonic transducer array 140 between two elevational positions along the z-axis. In FIG. 6A, the illustrated PACT system 600 is shown at an instant in time while a patient 10 is located on a bed/table 15 and the PACT system 600 is placed underneath the patient bed/table 15 with minimal separation from the top surface of the bed/table to the top scanning position of the ultrasonic transducer array 640.

FIG. 6B is a perspective view of components of the PACT system 600 partially shown in FIG. 6A. In FIG. 6B, the PACT system 600 is illustrated without the optical system 620 shown in FIG. 6A. As illustrated in FIG. 6B, the PACT system 600 includes a set of four 128-channel preamplifiers 650(1), 650(2), 650(3), and 650(4) in electrical communication with the 512-element ultrasonic transducer array 640 and a set of four 128-channel data acquisition systems (DAQs) 660(1), 660(2), 660(3), and 660(4) in electrical communication with the pre-amplifier(s) 650(1), 650(2), 650(3), and 650(4) respectively. Each of the DAQs is in communication with one of the preamplifiers. The set of four preamplifiers 650(1), 650(2), 650(3), and 650(4) and the set of four acquisition circuitry (DAQs) 660(1), 660(2), 660(3), and 660(4) are in one-to-one mapping association with the 512-element ultrasonic transducer array 640. The PACT system 600 also includes a computing device 680. Although not shown, the computing device 680 is in electrical communication (wired and/or wireless) with the (DAQs) 660(1), 660(2), 660(3), and 660(4) to receive signal(s) with photoacoustic data. In this illustrated example, a 512-element ultrasonic transducer array 640 is employed for panoramic acoustic detection. Four sets of 128-channel (DAQs) 660(1), 660(2), 660(3), and 660(4) provide simultaneous one-to-one mapped associations with the 512-element ultrasonic transducer array 640 to enable acquiring photoacoustic signals from a cross section within 100 μs without multiplexing after each laser pulse excitation. The ultrasonic transducer elements are unfocused and have a central frequency of 2.25 MHz and a one-way bandwidth of more than 95%. These ultrasonic transducer elements can provide an in-plane resolution of 255 μm. The height of each transducer element yields a diffraction angle (also referred to as divergence angle) in the elevational direction of about 9.0° full width at half maximum (FWHM), yielding a flared diffraction pattern. The unfocused transducer elements have diffraction angles that can provide elevational resolution in both 2D and 3D reconstruction. This pattern enables both 2D imaging of a breast cross section per laser pulse and 3D imaging of the whole breast by scanning elevationally. A 3D back projection algorithm described in Section III can be used to reconstruct a volumetric image with an elevational resolution of 5.6 mm, which is about 3 times finer than that given by the 2D reconstruction algorithm described in Section III. In another aspect, each unfocused transducer element in the array has a diffraction angle in a range of 5 degrees to 30 degrees. In another aspect, each unfocused transducer element in the array has a diffraction angle of about 20 degrees. In another aspect, each unfocused transducer element in the array has a diffraction angle in a range of 5 degrees to 15 degrees.

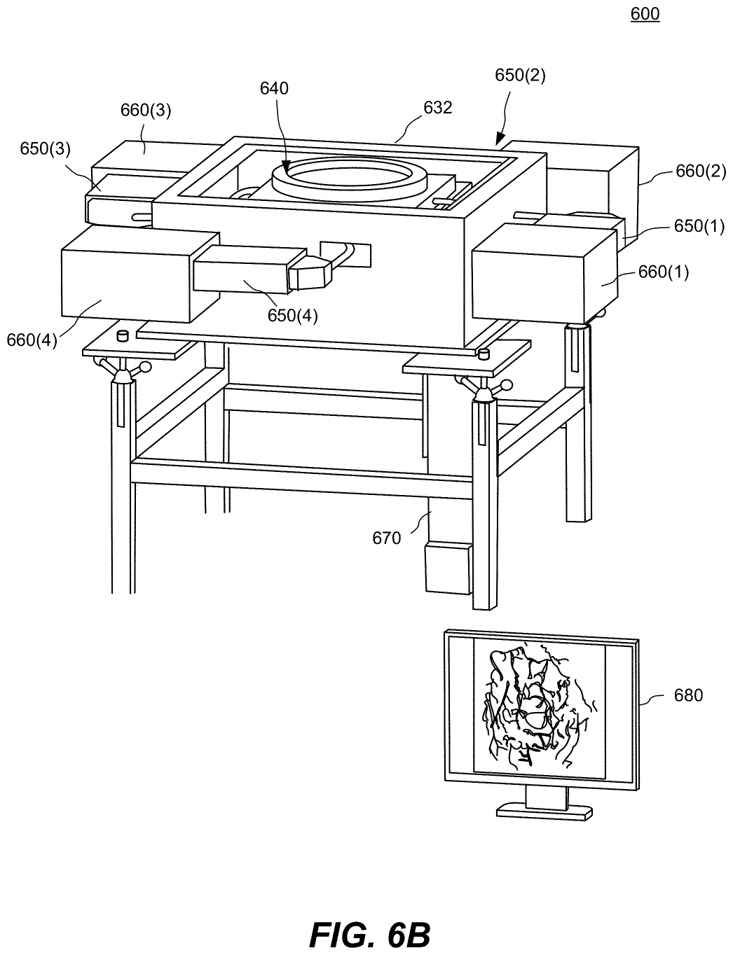

FIG. 7A is a perspective view of an example of a patient bed 730, according to an implementation. Although not shown, a PACT system is located underneath. The patient bed 730 includes a breast aperture 734 for receiving a breast of a patient while lying prone. FIG. 7B is a perspective, close-up view of a portion of the breast aperture 734 in the patient bed 730 shown in FIG. 7A. This illustrated example also shows a full ring ultrasonic transducer array 740.

Returning to FIGS. 6A and 6B, with the patient 10 lying prone on the bed/table 15, the breast 11 to be imaged is slightly compressed against the chest wall by a soft agar pillow. Compared to craniocaudal or mediolateral breast compression, compression against the chest wall not only avoids pain, but also gives the least thickness breast tissue for light to penetrate from the nipple to the chest wall. Once activated by trigger signal(s), the light source 610 illuminates the breast from beneath the bed/table 15, and the ultrasonic transducer array 640 detects photoacoustic waves circumferentially around the breast 11. The light beam is converted into a donut shape via the axicon lens 624 followed by the engineered diffuser 526. Compared to a Gaussian beam, the donut beam can provide more uniform illumination inside the breast and also deposit less energy on the nipple and areola, which have a higher concentration of pigment. A 1064 nm laser pulse from the light source 610 has low optical attenuation to achieve sufficient optical penetration (e.g., up to 4 cm) in breast tissue.

During operation, a 1064-nm laser beam from the light source 610 (e.g., PRO-350-10 made by Quanta-Ray with a 10-Hz pulse repetition rate and a 8-12-ns pulse width) is first reflected from the mirror 622, then passed through the axicon lens 624 (e.g., lab-polished axicon lens with 25 mm diameter and 160° apex angle), and then expanded by the engineered diffuser 626 (e.g., EDC-10-A-2s made by RPC Photonics) to form a donut-shaped light beam to circumferentially illuminate the breast 11. The laser fluence (e.g., 20 mJ/cm2) at the surface of the breast 11 in one example has been found to be within the American National Standards Institutes (ANSI) safety limit for laser exposure (i.e. 100 mJ/cm 2 at 1064 nm at a 10-Hz pulse repetition rate). In one aspect, to synchronize data acquisition with light pulses, the external trigger from the light source 610 may be used to trigger both the data acquisition systems 660 and the linear scanner 670.

The 512-element full-ring ultrasonic transducer array 640 (e.g., 512-element full-ring ultrasonic transducer array with 220 mm ring diameter and 2.25 MHz central frequency and more than 95% one-way bandwidth) is employed to provide 2D in-plane panoramic acoustic detection. Each transducer element had a flat-rectangular aperture (e.g., 5 mm element elevation size; 1.35 mm pitch; and 0.7 mm inter-element spacing). The ultrasonic transducer array housing was mounted on a stainless-steel rod (e.g., 25 mm diameter) and enclosed in the water tank 632. A linear scanner 670 (e.g., linear stage KR4610D made by THK America, Inc.) was fixed beneath the water tank 632 and moved the full-ring ultrasonic transducer array 640 elevationally via the stainless-steel rod. Four sets of 128-channel preamplifiers 650 (e.g., with 26 dB gain) were placed around the water tank 632, connected to the ultrasonic transducer array housing via signal cable bundles. Each set of preamplifiers 650 was further connected to a 128-channel data acquisition system 660 (e.g., SonixDAQ made by Ultrasonix Medical ULC with a 40 MHz sampling rate and 12-bit dynamic range) with programmable amplification up to 51 dB.

During operation of the PACT system 600 shown in FIGS. 6A and 6B, the digitized radio frequency data is first stored in an onboard buffer, and then transferred to the computing device 680 through a universal serial bus 2.0. The data acquisition systems 660 are set to record photoacoustic signals within 100 μs after each laser pulse excitation. During data acquisition, the patient 10 is positioned prone with the one breast 11 dependent and placed into a large aperture in the bed 15. An agar pillow may be affixed on top of an acrylic tube to lightly press the breast 11 against the chest wall. The bed top may be covered by cushioning memory foam. The water tank 632 may be fully filled with water preheated to a temperature of, e.g., 35° C. Both the patient bed 15 and the PACT system 600 may be supported by T-slotted aluminum frames.

The fours sets of 128-channel data acquisition systems 660 provide simultaneous one-to-one mapped associations with the 512-element transducer array 640 to acquire photoacoustic signals from a cross section within 100 μs without multiplexing after each laser pulse excitation. The ultrasonic transducer elements may have a central frequency of 2.25 MHz and a one-way bandwidth of more than 95%, providing in-plane resolution of 255 μm. The height of each transducer element in the 512-element transducer array 640 yields a divergence angle in the elevational direction of about 9.0° full width at half maximum (FWHM)), yielding a flared diffraction pattern. This flared diffraction pattern enables both 2D imaging of a breast cross section per laser pulse and 3D imaging of the whole breast by scanning elevationally. The 3D back projection algorithm in Section III can be used to reconstruct a volumetric image with an elevational resolution of 5.6 mm, which is about 3 times finer than that given by the 2D reconstruction algorithm described in Section III.

It would be understood that in FIGS. 6A and 6B and other illustrated examples, a PACT system is shown at an instant in time during operation where a specimen being imaged is located on a specimen receiving device at or near components of a PACT system during at least a data acquisition phase. At other instances, the specimen is not located at or near components of the PACT system.

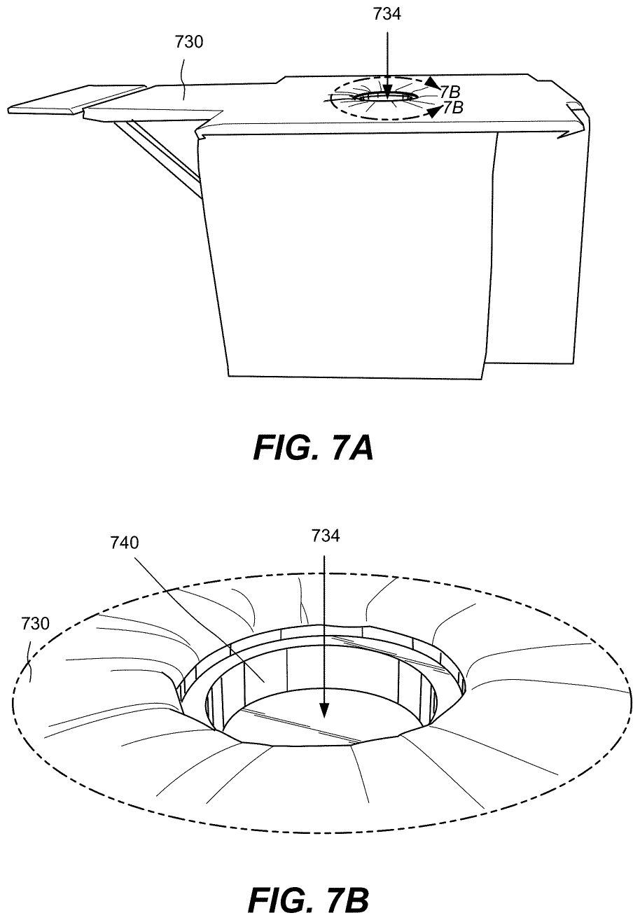

FIG. 8 is signal flow diagram of a PACT system 800, according to an embodiment. The PACT system 800 includes a light source 810 (e.g., a pulsed laser), an optical system (not shown that is configured to convert a light beam into shaped illumination such as donut-shaped illumination. The PACT system 800 also includes an ultrasonic transducer array 840 that can be coupled to or otherwise in acoustic communication with the specimen to receive photoacoustic signals induced by illumination. The PACT system 800 also includes one or more preamplifiers 850 and one or more data acquisition systems (DAQs) 860 in one-to-one mapped association with the transducers in the ultrasonic transducer array 840.

The one or more pre-amplifiers 850 are in electrical communication with the ultrasonic transducer array 840 to receive a signal or signals and the DAQ(s) 860 are in electrical communication with the pre-amplifier(s) 850 to receive a signal or signals. The PACT system 800 also includes a linear scanner 870 coupled to or otherwise operably connected to the ultrasonic transducer array 840 to move the ultrasonic transducer array 840 to one or more elevational positions and/or scan the ultrasonic transducer array 840 between two elevational positions. The PACT system 800 also includes a computing device 880 having one or more processors or other circuitry and a computer readable medium (CRM) in electronic communication with the processor(s). The PACT system 800 also includes a controller 885 in electronic communication with the DAQ(s) 860 and the linear scanner 870 to send control signals. To synchronize the PACT system 800, the light source's external trigger is used to trigger both the DAQ(s) 860 and the linear scanner 870. The electrical communication between system components of the PACT system 800 may be in wired and/or wireless form. The electrical communications may be able to provide power in addition to communicate signals in some cases. During operation, the digitized radio frequency data is first stored in an onboard buffer, and then transferred to the computing device 880, e.g., through a universal serial bus 2.0. The DAQ(s) 860 are configured to record photoacoustic signals within a time period, e.g., 100 μs, after each laser pulse excitation.

### III. Photoacoustic Computed Tomography (PACT) Methods

Certain aspects pertain to implementations of PACT systems and methods that can integrate deep penetration into biological tissues and high spatiotemporal resolution. In some cases, these PACT systems and methods may have potential to be useful in breast cancer detection.

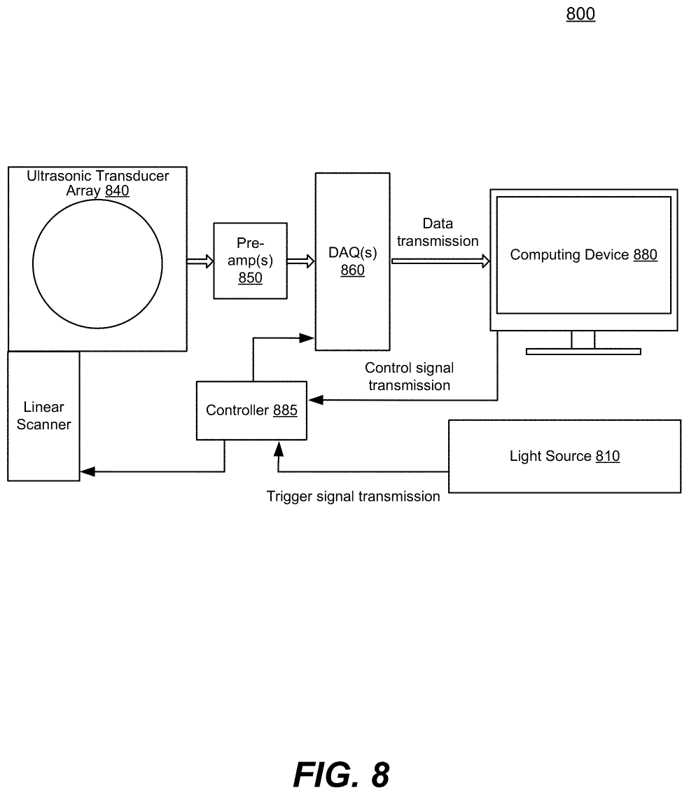

According to certain aspects, a PACT system is configured to be switchable between ( ) a two-dimensional (2D) mode; and (2) a three-dimensional ( 3D) mode. FIG. 9 is a flowchart depicting operations of a PACT method that can perform a 2D mode to obtain one or more 2D PACT images and/or a 3D mode to obtain at least one volumetric 3D image, according to certain aspects. The operations may be performed by, e.g., the PACT system 100 shown in FIG. 1 or the PACT system 600 shown in FIGS. 6A and 6B. One or more of the depicted operations are performed by executing instructions retrieved from memory. For example, a computing system may execute instructions retrieved from a CRM that causes control instructions for positioning the ultrasonic transducer array to be sent to a scanning mechanism coupled to the ultrasonic transducer array.

At operation 910, the PACT system controls system components to perform data acquisition in a 2D mode or a 3D mode. Alternatively, data acquisition may be in both modes consecutively, e.g., in the 2D mode and then the 3D mode or in the 3D mode and then the 2D mode. The PACT system synchronizes data acquisition by the DAQ(s) and pre-amplifiers with the light pulses from the light source to acquire photoacoustic signals from the illuminated specimen. In one aspect, to synchronize data acquisition with light pulses, the external trigger from the light source may be used to trigger both the data acquisition systems and the scanning mechanism.

During data acquisition in the 2D mode, the ultrasonic transducer array is moved to one or more elevational positions (e.g., different locations z₁, z₂, z₃, z₄, etc. along a z-axis in FIG. 6A) and held in each elevational position for a time period. Some examples of time periods that can be used include about 10 seconds, about 15 seconds, and about 20 seconds. In one case, the time period is in a range of about 10 seconds to about 20 seconds. At each cross section, photoacoustic signals are continuously recorded at a certain sampling rate to monitor the cross section. For example, the ultrasonic transducer array may be moved so that the ultrasonic transducer array is located (e.g., center of each unfocused transducer located) approximately at four different elevational positions z₁, z₂, z₃, and z₄. The elevational positions z₁, z₂, z₃, and z₄ may be selected so that the separation between the elevational positions corresponds to the elevational resolution of 2D reconstructed image for the ultrasonic transducer array. For example, if the elevational resolution in 2D reconstructed image is 2 cm or a particular ultrasonic transducer array, then the separation between the elevational positions may be selected as 2 cm or less. In one case, the separation is 1 cm and to obtain 2D images through a depth of 4 cm through a human breast, elevational positions of z₁=0; z₂=1 cm; z₃=2 cm; and z₄=3 cm may be selected. The separation of 1 cm between the depths is selected since it is less than the elevational resolution in 2D for the ultrasonic transducer array being used. Some examples of suitable sampling rates include 10 Hz, between 20 and 25 Hz, and about 24 Hz. If a mass detection procedure with an elastography study will be conducted at operation 950, data acquisition in 2D mode will be conducted while the specimen is deforming in order to continuously monitor the deformation. For example, a human patient may be allowed to breathe during data acquisition to allow a breast to deform. By holding the ultrasonic transducer array at a specific elevational position, the PACT system can continuously monitor arterial pulsatile deformation inside the breast, particularly through the depth of the elevational resolution of the unfocused transducer elements. In one example, the time period is 10 seconds for each of four cross sections and the sampling rate for recording the photoacoustic signals is 10 Hz for the four depths separated by 1 cm (e.g., at z₁=0; z₂=1 cm; z₃3=2 cm; and z₄=3) through the biological tissue. In this example, 100 2D images will be acquired for each of the four cross-sections. During each of these four 10-second time periods, the patient breathes normally while the photoacoustic signals are recorded. A separation of 1 cm is selected in this case since it is less than the elevational resolution in 2D of 1.61 cm for the ultrasonic transducer array being used.

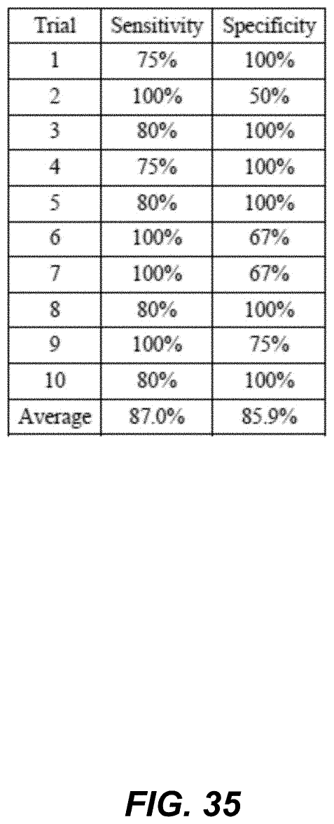

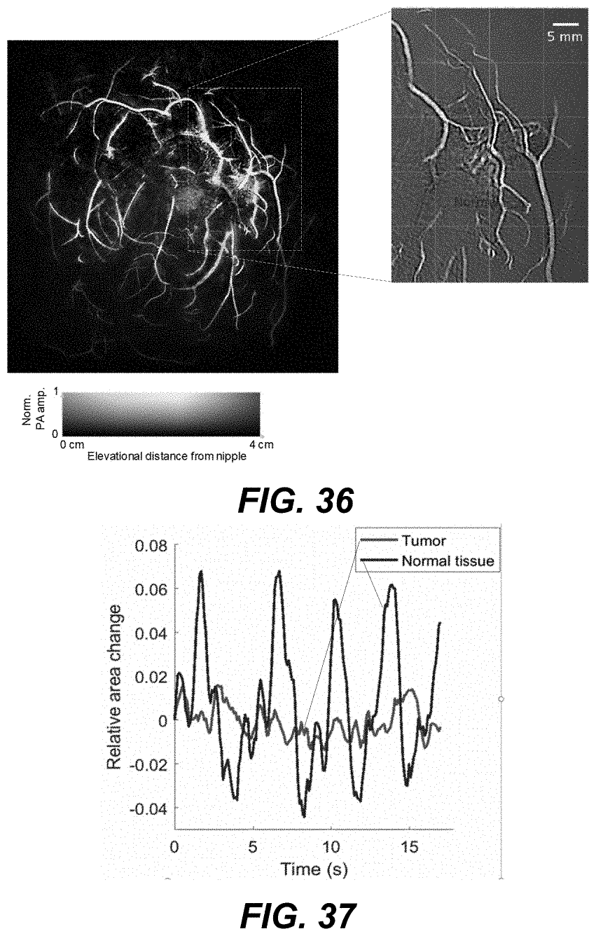

FIGS. 36 and 37 illustrates an elastographic evaluation of a cancerous breast using a PACT method. FIG. 36 is a PACT image of a 69-year-old female patient with an invasive ductal carcinoma of grade 2/3, according to an implementation. FIG. 37 is a plot of the relative area change over time for both the tumor and the normal tissue, according to an implementation. As shown, the tumor changes relative area to a lesser degree than the normal tissue.

During acquisition in the 3D mode, the ultrasonic transducer array is scanned through multiple scanning steps between two elevational positions through a depth (e.g., through a depth between z₁ and z₂ locations along a z-axis in FIG. 6A). For example, the ultrasonic transducer array may be moved so that the center of each unfocused transducer element in a ring is scanned through multiple scanning steps between two elevational positions z₁ and z₂. In one aspect, when imaging a human breast in 3D mode, the ultrasonic transducer array may be controlled to scan the entire breast from the chest wall to the nipple. In some cases, the breast has a depth between the chest wall and the nipple of 4 cm or is compressed to be within this depth of 4 cm. In this case, the depth of the volumetric 3D image is 4 cm. In one example, a volumetric 3D image having a depth of 4 cm through the human breast is taken using elevational positions of z₁=0; z₂=4 cm. For a single-breath-hold scanning to image a breast (e.g., 4-cm thick), the scan may be about 5 cm. Using the data collected along the 5 cm, a 4-cm thick image can be reconstructed. The elevational scanning data can be used to reconstruct an image at any thickness. In certain instances, the elevational scanning distance of the array is longer than the reconstructed image's thickness.

The photoacoustic signals are recorded at a certain sampling frequency, which is determined by the data acquisition circuits. In one example, the sampling frequency is 40 MHz. In one aspect, the sampling frequency can be in a range from 4 MHz to 80 MHz. The time-domain photoacoustic signals acquired at all elevational scanning steps may then back-projected simultaneously into the 3D space. If tumor segmentation is going be performed operation 950, data acquisition in 3D mode may be conducted while the specimen is still to try to avoid any motion artifacts. For example, a human patient may be asked to hold their breathe during data acquisition.

At operation 920, the photoacoustic signals are received by the computing device from the DAQ(s). In some cases, the PACT system is equipped with a one-to-one mapped signal amplification and data acquisition (DAQ) systems or DAQ circuits to the transducer elements. In these cases, the PACT system can obtain photoacoustic signals for a 2D cross-sectional image with each laser pulse in 2D mode or obtain photoacoustic signals for a volumetric 3D image (e.g., of an entire breast) by fast elevational scanning within the time period such as, e.g., a single breath-hold (about 15 sec).

At operation 930, the photoacoustic signals are low-pass filtered with cut-off frequencies determined by the maximum distance from a point in the specimen being imaged to the transducer elements. For example, if a full-ring transducer array with 512 elements is used, the array can spatially sample objects within a field of view (FOV) of about 39 mm according to the spatial Nyquist criterion. To eliminate aliasing caused by under-sampling in regions outside of this FOV, the photoacoustic signals may be low-pass filtered with cut-off frequencies determined by the distance to the center of the ring array.

At operation 940, the PACT system performs image reconstruction to: 1) reconstruct a plurality of 2D images for each elevational position of the ultrasonic transducer array taken over a time period (2D mode) and/or 2) reconstruct a volumetric 3D image for the depth scanned by the ultrasonic transducer array ( 3D mode). In one aspect, a universal back-projection process can be used to reconstruct one or more 2D/3D images. An example of a universal back-projection process can be found in Xu, M. And Wang, L., “Universal back-projection algorithm for photoacoustic computed tomography,” Physical Review E 71, 016706 (2005), which is hereby incorporated by reference in its entirety. Another example of a back-projection process can be found in Anastasio, M. A. et al., “Half-time image reconstruction in thermoacoustic tomography,” IEEE Trans., Med. Imaging 24, pp 199-210 (2005), which is hereby incorporated by reference in its entirety. In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S patent application 2019/0307334, titled “SINGLE-IMPULSE PANORAMIC PHOTOACOUSTIC COMPUTED TOMOGRAPHY” and filed on May 29, 2019, which is hereby incorporated by reference in its entirety.

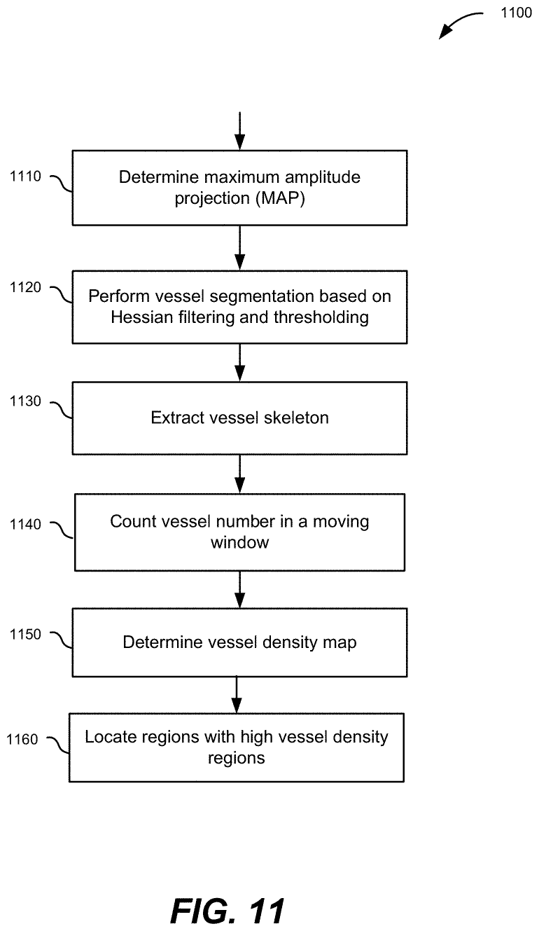

In one implementation, the half-time universal back-projection (UBP) process was used to reconstruct a volumetric 3D image and a 2D image of a breast using the PACT system 600 shown in FIGS. 6A and 6B. An example of a half-time UBP process is discussed in Anastasio, M. A. et al., “Half-time image reconstruction in thermoacoustic tomography,” IEEE Trans. Med. Imaging 24, 199-210 (2005), which is hereby incorporated by reference in its entirety. In the 2D imaging mode, the time-domain PA signals generated by each laser pulse were back-projected to a 2D imaging plane. Determined by the acoustic divergence angle (about) 9.0° at FWHM in the elevational direction as shown in FIG. 3A, the elevational resolution at the center was about 16.1 mm. Alternatively, when working in 3D mode, the ultrasonic transducer array scanned the entire breast from the chest wall to the nipple. The time-domain PA signals acquired at all elevational scanning steps were then back-projected simultaneously into the 3D space. To accommodate the acoustic divergence angle in the elevational direction, the UBP process added a weight to the back-projected photoacoustic signals at different elevational divergence angles. To accurately reconstruct objects in the Fraunhofer zone, the photoacoustic signals were back-projected from virtual transducers located at the transition points between the Fresnel and Fraunhofer zones. Sharing the same in-plane resolution as the 2D mode, the UBP process provided an improved elevational resolution of 5.6 mm. The elevational resolution of the volumetric 3D image was 5.6 mm, which is about 3 times finer than the elevational resolution of the 2D image. An example of a UBP process is described with respect to the flowchart shown in FIGS. 12A and 12B.

The 3D volumetric image is reconstructed with a particular voxel size in both the elevational direction and in the horizontal plane. An example of a suitable voxel size in the elevational direction is about 1 mm. An example of a suitable voxel size in the horizontal plane is 0.1 and 0.1 mm². In some cases, one or more of the reconstructed images are batch processed to improve contrast. For example, a 3D volumetric image may be batch processed using vesselness filtering to improve contrast of blood vessels. An example of vesselness filtering that can be used is Hessian-based Frangi vesselness filtration described in Li, L. et al., “Single-impulse panoramic photoacoustic computed tomography of small animal whole body dynamics at high spatiotemporal resolution,” Nat. BME 1, 0071 (2017), which is hereby incorporated by reference in its entirety. In one implementation, e.g., in each horizontal slice of a 3D volumetric image, Hessian-based Frangi vesselness filtration was applied to enhance the contrast of blood vessels with diameters ranging from 3 to 12 pixels.

Returning to FIG. 9, at operation 950, the PACT system optionally (denoted by a dotted line) performs a tumor detection procedure. In this case, the images reconstructed are of biological tissues. The tumor detection procedure is implemented to identify any masses of interest in the imaged biological tissue that may potentially be tumors. The tumor detection procedure may be performed in a 2D mode or a 3D mode. Alternatively, tumor detection procedure may be in both modes consecutively, e.g., in the 2D mode and then the 3D mode or in the 3D mode and then the 2D mode. For example, the 3D tumor detection procedure may be performed first and if there is a questionable mass of interest, the tumor detection procedure in 2D may performed.

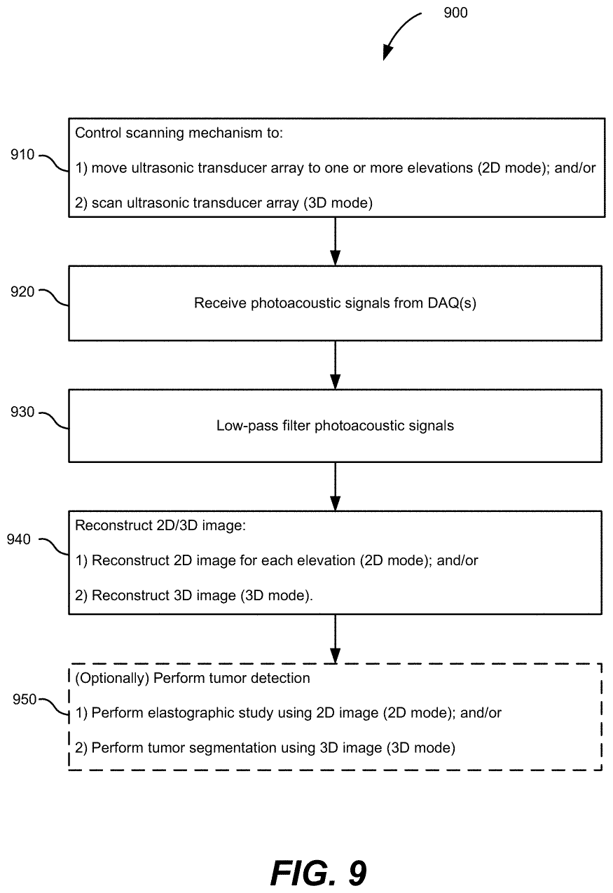

In the 2D mode, the mass detection procedure includes performing an elastographic study (evaluation) on a plurality of 2D photoacoustic images. The high imaging speed of the PACT system allows for differentiation in compliance (or stiffness) between tumors and surrounding normal tissue. Tumors tend to be less compliant, deforming to a lesser extent, than surrounding normal tissue. The PACT method can differentiate between tumors and surrounding normal tissue by analyzing the differential compliance in the images taken at high speed of a cross-section. An example of operations in a mass detection procedure for this 2D mode is described in detail with reference to FIG. 10. This differential compliance can be used as another contrast for detecting breast cancer.

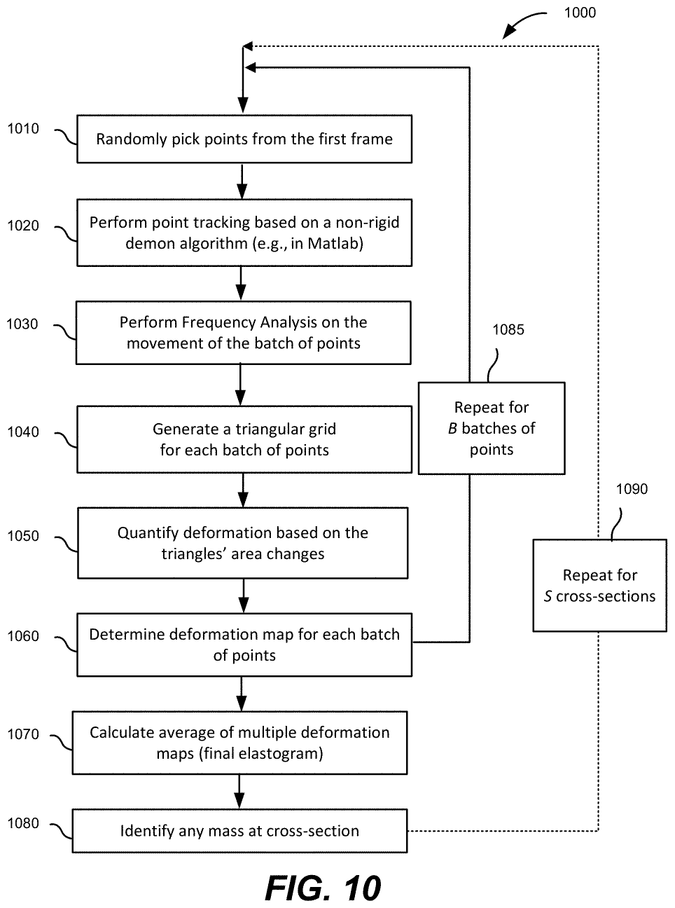

In the 3D mode, the mass detection procedure includes tumor segmentation of a volumetric 3D image taken by the PACT system. An example of operations in a tumor detection procedure for the 3D mode is described with reference to FIG. 11.

FIG. 12A is a flowchart of operations of a universal back-projection process that can be used to reconstruct either a 2D image or a 3D image, according to an implementation. FIG. 12B is a flowchart of additional operations of the universal back-projection process in FIG. 12A as used for the 3D image, according to an implementation. More details for an example of this process can be found in Anastasio, M. A. et al., “Half-time image reconstruction in thermoacoustic tomography,” IEEE Trans. Med. Imaging 24, 199-210 (2005), which is hereby incorporated by reference in its entirety.

At operation 1210, photoacoustic signals are received, e.g., from the data acquisition systems. The photoacoustic signals are based on the photoacoustic array being at one location while photoacoustic waves are being detected. At operation 1220, a low-pass filter is applied to the photoacoustic signals to remove noise. For example, a low-pass filter of 4.5 MhZ may be used. For a pixel and an element location, the time delay is calculated at operation 1230. At operation 1240, an acquired signal at the calculated time delay is used to calculate the back-projection term and this is added to the pixel value. At operation 1260, the process returns to repeat operations 1230 and 1240 for all combinations of pixel and element locations. At operation 1250, a 2D image is formed of all the pixel values.

A similar set of operations is depicted for reconstructing a 3D image. That is, at operation 1210, photoacoustic signals are received, e.g., from the data acquisition systems. In this case, the photoacoustic signals are based on the photoacoustic array being scanned between two positions while photoacoustic waves are being detected. At operation 1220, a low-pass filter is applied to the photoacoustic signals to remove noise. For example, a low-pass filter of 4.5 MhZ may be used. For a voxel and an element location, the calculated time delay is calculated at operation 1230. At operation 1240, an acquired signal at the time delay is used to calculate the back-projection term and this is added to the voxel value. At operation 1260, the process returns to repeat operations 1230 and 1240 for all combinations of voxel and element locations. At operation 1250, a 3D image is formed of all the voxel values.

In FIG. 12B, the 3D image from operation 1250 in FIG. 12A is received. At operation 1260, in the elevational direction of each filtered volumetric 3D image, voxels are selected with the largest photoacoustic amplitudes to form a maximum amplitude projection (MAP) 2D image in grayscale. At operation 1270, the depths of the voxels along with their largest photoacoustic amplitude values from operation 1260 are used to form a depth map in grayscale. At operation 1280, the grayscale depth map is transferred to a colorful depth image. At operation 1290, the MAP image is used to modulate the colorful depth image in three channels (RGB), resulting in a color-encoded MAP image. A median filtration with a window size of 3×3 pixels to the depth image. Another median filtration with a window size of 6×6 pixels was further applied inside the segmented vessels to the segmented vessels' depths. Different RGB (red, green, blue) color values were assigned to discrete depths. The 2D depth-resolved color-encoded image was multiplied by the MAP image pixel by pixel to represent the maximum amplitudes. In one aspect, to further reduce noise and improve image quality, the parameters were tuned in 2D slices at different depths. In one example, the structures in all three sets of images match well with each other, showing the fidelity of the vesselness filtering and custom processing.

### A. Methods for Identifying Masses of Interest

Certain aspects pertain to methods that may be used to identify masses of interest in, e.g., biological tissues, using either a plurality of 2D images of a cross section or a volumetric 3D image. For example, one aspect pertains to methods that may be used to identify masses using elastographic measurements from a plurality of 2D images of a particular cross-section acquired at high speed over a period of time. As another example, another aspect pertains to a method of identifying masses using a quantified density of blood vessels counted in regions of a volumetric 3D image.

One aspect pertains to a PACT method that uses a plurality of 2D images reconstructed from photoacoustic signals recorded at high imaging speed (e.g., at or above 10 Hz frame rate) at each of one or more cross-sections (depths). During data acquisition, a plurality of 2D images is acquired at high speed at each of the depths while the specimen is allowed to deform (e.g., small deformations less than or equal to 1 cm). For example, a patient may breathe normally while photoacoustic signals are recorded while an ultrasonic transducer array of the PACT system 600 in FIG. 6A and FIG. 6B detects photoacoustic waves at each of one or more depths of a breast. This PACT method takes elastographic measurements at each of the one or more cross-sections. Tumors, being stiffer than normal tissue, can be identified in regions with less deformation than the normal tissue. The high-speed imaging speed enables differentiation in compliance between tumors and normal tissue. This PACT method may use the elastographic measurements at each of the one or more cross-sections to identify masses of interest based on deformations determined at the one or more cross-sections. This PACT method may be used to differentiate between breast tumors and surrounding normal tissue, which may potentially provide another technique for detecting breast cancer.

Another aspect pertains to a PACT method that uses a volumetric 3D image reconstructed from photoacoustic signals recorded while the specimen remains still and the ultrasonic transducer array scans through the depth. For example, in the 3D data acquisition mode, patients may hold their breath while photoacoustic signals are recorded as during a scan of a human breast from the breast wall to the nipple. As the principal optical absorber in the near infrared region, hemoglobin provides an endogenous contrast for imaging of blood vessels. A high density of blood vessels tends to correlate with angiogenesis, which may play an important role in tumor growth and metastasis. This second PACT method includes an automated segmentation process that extracts a vessel skeleton from the volumetric 3D image, produces a vessel density (number of vessels/area) map of the biological tissue such as a breast and then highlights a region with highest vessel density as a mass of interest. Due to angiogenesis in tumor regions, this second PACT method may be used to show masses of interest by revealing a greater density of blood vessels in certain regions.

### Method 1 with Elastographic Measurements (2D mode)

FIG. 10 is a flowchart of operations of an exemplary mass detection method that performs elastographic evaluation of a plurality of 2D images acquired over a time period for each cross-section of a set of one or more cross-sections, according to certain implementations. The 2D images may be acquired by a PACT system or other imaging system. For example, the PACT system 100 of FIG. 1 can be used to acquire a plurality of 100 2D images, for each of four cross-sections at four different depths of a breast, during a time period of 10 seconds at a frame rate of 10 Hz while the patient is breathing. For simplicity, the operations of the exemplary mass detection method are described with reference to frames acquired of a human breast, it would be understood that other this method can also be used to perform elastographic evaluation on other deforming specimens.

At operation 1010, to assess deformations over time, the first 2D image (frame) is taken as a reference and a batch of points (pixels) is randomly picked from the first 2D image.

At operation 1020, movement of the batch of points is tracked using a tracking process that registers the other frames with the first frame. An example of a tracking process is a non-rigid demon process, e.g., the non-rigid demon function in Matlab. An example of a tracking process is also described in Thirion, J P., “Image matching as a diffusion process: an analogy with Maxwell's demons,”Med. Image Anal. 2, 243-260 (1998), which is hereby incorporated by reference in its entirety. The non-rigid demon process defines a feature around each point in the batch of points to determine its movement. For each point of the registered frames, the standard deviation (STD) of the value variations was calculated. Points with relatively small STDs (e.g., less than a maximum allowable STD) were stably registered and were used for deformation quantification. The other points with large STDs were removed. An example of a maximum allowable STD is 0.18.

At operation 1030, the movement of the batch of points is analyzed in the frequency domain and the high frequency movements, which are generally not due to breathing, are removed by low-pass filtering. The frequency component due to deformation from breathing is in a range of 0.2-0.5 Hz. The small and/or high frequency movements are removed by filtering so that mainly movements due to breathing are being monitored. At operations 1020 and 1030, small and/or high frequency movements are removed so that movements due to breathing with larger and lower frequency are being monitored.

At operation 1040, a triangular grid for the batch of points is generated to be able to track deformation of areas. The triangular grid includes a plurality of triangles formed from the batch of points. In some cases, the triangles may share points. The triangular grid is mapped back to the unregistered frames and their triangular areas (areas of the triangles) are calculated. In one case, the triangle grid may be generated, for example, using a Matlab function. The entire image was then segmented into 2 mm×2 mm squares. One stably registered pixel was chosen from each square, and triangular grids were further generated from these registered pixels.

At operation 1050, the deformation based on changes to the areas of the triangles in the triangular grid is calculated. The tracked movement of the points of each triangle is used to determine the deformation of the area of each triangle in the triangular grid.

At operation 1060, a deformation map for each batch of points is determined. The deformation map includes the changes in area of each of the triangles. For each triangle, Fourier transformation was applied to quantify the area variation at the frequency of periodic compression, and amplitudes were assigned to the points of the triangle to generate the deformations for the deformation map. For example, the amplitude of the deformation of each triangle may be mapped to the points of the triangle to generate the deformation map.

At operation 1085, the process returns to repeat operations 1010, 1020, 1030, 1040, 1050, and 1060 for B batches of points where B is the number of batches of points (e.g., B may be 100).

After operations 1010, 1020, 1030, 1040, 1050, and 1060 have been completed for each of the batches of points to determine a plurality of B deformation maps, an average of the plurality of deformation maps is calculated to determine a final elastogram (operation 1070). For example, at operation 1070, the average deformation for all the deformation maps may be determined for each point in the batches of points and mapped to that point to determine a final elastogram.

At operation 1080, the final elastogram is evaluated to identify any region at the cross section with a potential mass. For example, deformation at different points in the elastogram may be evaluated to determine whether the deformation at any of the points is below a threshold value. The location of the point that is below a threshold value may be determined to be in a region potentially having a mass. An example of a threshold value is 0.036. Another example of a threshold value is 0.048. As another example, if the deformations of multiple neighboring points are below a threshold value, it may be determined that potentially there is a mass in the region of the neighboring points.

At operation 1090, the process repeats for each additional cross-section for S cross-sections (e.g., S=3, 4, 5, or 6). For example, if photoacoustic signals are acquired for each of four cross-sections at four depths (e.g., for depths separated by 1 cm, which is less than the elevational resolution in 2D for the ultrasonic transducer array), then the process will repeat four times. Alternatively, the process will be performed for all cross sections in parallel. If only one cross-section is being evaluated, the process is performed once and operation 1090 can be omitted.

### Method 2 with Automated Mass Segmentation ( 3D Mode)

FIG. 11 is a flowchart of operations of an exemplary mass detection procedure that performs an automated mass segmentation process of a volumetric 3D image acquired in 3D mode, according to one aspect.

At operation 1110, the maximum amplitude projection (MAP) is determined at each pixel of the volumetric 3D image. First, the nipple layers of the volumetric 3D are removed. Each voxel at different depths of the volumetric 3D image is evaluated to determine the voxel with the maximum amplitude. The voxels with maximum amplitude are projected to a plane to generate a MAP.

At operation 1120, vessel segmentation is performed on the 3D volumetric image using vesselness filtering and thresholding. The vesselness filtering process can improve contrast of any blood vessels in the 3D image. In one implementation, in each horizontal slice, a vesselness filtering process is applied in each horizontal slice of the 3D volumetric image. An example of vesselness filtering process that can be used is Hessian-based Frangi vesselness filtration. Hessian-based Frangi vesselness filtration is described in Li, L. et al., “Single-impulse panoramic photoacoustic computed tomography of small animal whole body dynamics at high spatiotemporal resolution,” Nat. BME 1, 0071 (2017), which is hereby incorporated by reference in its entirety. For example, in each horizontal slice of the 3D volumetric image, the vesselness filtration process may be applied to enhance the contrast of blood vessels with diameters ranging from 3 to 12 pixels. In this example, the voxel has a size of 1 mm in the elevational direction and 0.1×0.1 mm² on the horizontal plane. After the vesselness filtering process is applied, adaptive thresholding is used for each filtered horizontal slice to segment blood vessels. An example of counting blood vessels is described in Tsai, P. S. et al., “Correlations of neuronal and microvascular densities in murine cortex revealed by direct counting and colocalization of nuclei and vessels,” J. Neurosci. 29, 14553-14570 (2009), which is hereby incorporated by reference in its entirety.

At operation 1130, a vessel skeleton is extracted. The vessel skeleton includes all the vessels segmented in operation 1120. In the vessel skeleton, the vessels have been turned into lines. For example, a vessel skeleton may be extracted by using morphology filtration for single-pixel elimination.

At operation 1140, the vessels in a moving window are counted for each window position to determine vessel numbers for each window in the MAP of a 3D image. An example of the window size is 15×15 pixels. Another example of the window size is 20×20 pixels. Other window sizes would be contemplated. In one example, the window movement may be two pixels in one direction for each window position. In another example, the window movement may be three pixels in one direction for each window position.

At operation 1150, a vessel density map is determined. At each window position, the density is calculated using the numbers of vessels counted at each window position and the area of the corresponding window. The density map includes the calculated densities for different pixel locations at the window positions across each horizontal slice in the 2D MAP image.

At operation 1160, one or more regions with high vessel density are located. For example, a threshold vessel density value may be calculated and any pixels with vessel density greater than the threshold vessel density value may be determined to have a high vessel density. In one implementation, the threshold vessel density value may be a set value. In one example, the threshold vessel density value is in the range of whole-breast's average plus 1.0 time the standard deviation to 2.0 times the standard deviation. In another example, the threshold vessel density value is above whole-breast's average plus 2.0 times the standard deviation. In one implementation, the vessel density value is set by an operator. In another implementation, the vessel density value is calculated from a maximum vessel density in the 3D volumetric image. For example, a threshold vessel density value may be 90% of the maximum vessel density in the 3D volumetric image. These regions may be designated as potential masses of interest in one implementation.

### B. Method 3—Vascular Diameter Measurement (2D or 3D mode)

In one embodiment, the process described with reference to FIG. 11 further includes measuring vascular diameters of the vessels by identifying vessel boundaries in different slices of a 3D volumetric image or in a 2D image using a correlation-based template matching method. An example of such a correlation-based template matching method is described in Tsai, P. S. et al., “Correlations of neuronal and microvascular densities in murine cortex revealed by direct counting and colocalization of nuclei and vessels,” J. Neurosci. 29, 14553-14570 (2009), which is hereby incorporated by reference in its entirety. The templates may be generated through simulation in some cases. The impulse responses of all ultrasonic transducers can be used to simulate the images of vessels with different sizes (e.g., in a range of 0.5 mm to 2.0 mm) and orientations. The diameters of vessels chosen from the images can be measured by matching the reconstructed vessel images with the generated templates.

### C. Method 4—Arterial Vessel Mapping (2D mode)

In one embodiment, the process described with reference to FIG. 10 further includes arterial vessel mapping operations. This arterial vessel mapping can be used to monitor blood flow-mediated arterial fluctuation. After removing displacement through rigid transformation using the non-rigid demon process at operation, the pixel value fluctuation is analyzed during the time period such as a patient's breath hold of about 10 seconds or about 15 seconds. Arteries may fluctuate more than veins at the frequency of the heartbeat. The fluctuation of the pixel values in the artery indicated the changes associated with arterial pulse propagation. To separate fluctuations caused only by heart beats, frames with strong motion caused by body movement were first removed. The entire imaging field was then divided into n slightly overlapping subdomains (e.g., n=16). In each subdomain, the first frame was selected as the reference frame and other frames were registered to it through rigid transformation, optimizing the frame-frame correlation. In each subdomain, a Gaussian filter with a certain radius (e.g., 0.2 mm) was applied to all registered frames to reduce high spatial-frequency noise. A Fourier transformation was applied to each pixel's value through all the frames. The fluctuations in pixel values induced by arterial pulse propagation were quantified within the frequency range (1.0-1.6 Hz) of heartbeat cycles.

### D. Some Exemplary Methods for Identifying Breast Tumors

Two exemplary PACT methods that can be used to identify one or more regions of potential masses that may be breast tumors in angiographic photoacoustic computed tomography (PACT) images are provided below. In Section IV, evaluation data from employing examples of these two methods to image seven breast cancer patients has been provided. In the evaluation case, the ability to detect tumors was demonstrated via blood vessel density using a PACT method with automated tumor segmentation in eight of nine cases, and using a PACT method with elastography to detect differences in the stiffness of the tissue in the remaining case.

### 1. Method with Automated Tumor Segmentation Technique

Certain implementations of PACT methods employ an automatic tumor segmentation technique that may make it easier to recognize a tumor by highlighting a region with high vessel density. Due to angiogenesis in tumor regions, PACT images may be used to identify breast masses by revealing a greater density of blood vessels. To segment tumors automatically, a vessel skeleton may be extracted and a vessel density (number of vessels/area) map of the breast determined. The regions with the highest vessel density highlight regions with potential breast masses. FIG. 11 illustrates an example of operations that can be used to implement this method.

### 2. Method with Elastography

High speed imaging such as available with PACT systems enables capturing images that can be used to differentiate compliance between tumors and surrounding normal breast tissue, providing another contrast for detecting breast cancer. During image acquisition, patients are asked to breathe normally. The chest wall pushed the breast against the agar pillow, elevationally generating a deformation of the breast in the coronal plane. The change of area at different points are determined. Tumors, being stiffer, could be identified in areas with less deformation than normal breast tissue. FIG. 10 illustrates an example of operations that can be used to implement this method.

The American Cancer Society recommends regular examinations of breast lesions as the best way to detect breast cancers early. The automatic tumor segmentation technique of certain implementations may make it easier to recognize tumors by highlighting a region with high vessel density. In addition, the high 2D imaging speed (e.g., 10 Hz frame rate) of certain implementations of PACT systems can enable performing elastographic measurements and further improve on breast cancer detection. Moreover, PACT systems are different from mammography in that PACT systems do not implement ionizing radiation and do not have the limitations in radiographically dense breasts. As compared to MRI, PACT systems do not use exogenous contrast agents and can scan an entire breast within a single breath hold of about 15 seconds.

### IV. Examples

The non-limiting examples provided in Section IV are to further illustrate certain implementations of PACT techniques. It would be appreciated that certain techniques implemented in these examples represent approaches for PACT systems and methods that have been found to function well, and thus can be considered to constitute examples of modes for their practice. However, those of skill in the art should, in light of the present disclosure, appreciate that many changes can be made in the specific examples that are disclosed and still obtain a like or similar result without departing from the spirit and scope of the present disclosure.

### A. Examples of a PACT Systems and PACT Methods

An example of a PACT system was used to image the breasts of one healthy volunteer and seven breast cancer patients. The PACT system included an illumination (light) laser source, an ultrasonic transducer array, signal amplification/acquisition modules, a linear scanning stage, and a patient bed similar to the PACT system 600 described with reference to FIGS. 6A and 6B. The light source used was a 1064 -nm pulse laser source (e.g., PRO-350-10, Quanta-Ray, 10-Hz pulse repetition rate, 8-12-ns pulse width). The 1064-nm laser beam was first passed through lab-polished axicon lens (25 mm diameter, 160° apex angle), then expanded by an engineered diffuser (EDC-10-A-2s, RPC Photonics) to form a donut-shaped light beam. The laser fluence (20 mJ/cm 2) was within the American National Standards Institutes (ANSI) safety limit for laser exposure (100 mJ/cm 2 at 1064 nm at a 10-Hz pulse repetition rate) To synchronize the PACT system, the laser's external trigger was used to trigger both the data acquisition systems and the linear scanner.

To achieve 2D panoramic acoustic detection, a 512-element, full-ring ultrasonic transducer array (e.g., Imasonic, Inc.; 220 mm ring diameter; 2.25 MHz central frequency; more than 95% one-way bandwidth) was used. The transducer elements were unfocused and had a central frequency of 2.25 MHz and a one-way bandwidth of more than 95%. Each transducer element had a flat-rectangular aperture (5 mm element elevation size; 1.35 mm pitch; 0.7 mm inter-element spacing). The ultrasonic transducer array housing was mounted on a stainless-steel rod (25 mm diameter) and enclosed in an acrylic water tank. A linear stage (e.g., THK America, Inc., KR 4610D) was fixed beneath the water tank and moved the transducer array elevationally via the stainless-steel rod. The ultrasonic transducer array had an in-plane resolution of 255 μm as described in FIGS. 5A and 5B. The height of each unfocused transducer element yielded a divergence angle in the elevational direction of about 9.0° full width at half maximum (FWHM).

Four sets of 128-channel preamplifiers (26 dB gain) were placed around the water tank, connected to the ultrasonic array housing via signal cable bundles. Each set of preamplifiers was further connected to a 128-channel data acquisition system (e.g., SonixDAQ, Ultrasonix Medical ULC; 40 MHz sampling rate; 12 -bit dynamic range) with programmable amplification up to 51 dB. The digitized radio frequency data were first stored in an onboard buffer, and then transferred to a computer through a universal serial bus 2.0. The data acquisition systems were set to record PA signals within 100 μs after each laser pulse excitation. This PACT system was equipped with four sets of 128-channel data acquisition systems to provide simultaneous one-to-one mapped associations with the 512-element transducer array. Therefore, photoacoustic signals were acquired from a cross-section within 100 μs without multiplexing after each laser pulse excitation.

During the data acquisition phase, the patient being imaged was positioned prone with one breast dependent and placed into a large aperture in the bed such as the patient bed 15 shown in FIG. 7A. An agar pillow affixed on top of an acrylic tube lightly pressed the breast against the chest wall. The bed top was covered by cushioning memory foam. The water tank was fully filled with water preheated to a temperature of 35° C. Both the patient bed and the PACT system were supported by T-slotted aluminum frames.

A PACT method of an implementation employed a half-time universal back-projection (UBP) process to reconstruct a 3D volumetric image and a plurality of 2D images of a cross-section acquired over a time period. An example of a half-time UBP process can be found in Anastasio, M. A. et al., “Half-time image reconstruction in thermoacoustic tomography,” IEEE Trans. Med. Imaging 24, 199-210 (2005), which is hereby incorporated by reference in its entirety. In 2D imaging mode, the time-domain photoacoustic signals generated by each laser pulse were back-projected to a 2D imaging plane. Determined by the acoustic divergence angle (about) 9.0° at FWHM in the elevational direction discussed in FIGS. 3A-3C, the elevational resolution at the center was about 16.1 mm. Alternatively, when working in 3D mode, the ultrasonic transducer array scanned the entire breast from the chest wall to the nipple. The time-domain photoacoustic signals acquired at all elevational scanning steps were then back-projected simultaneously into the 3D space. To accommodate the acoustic divergence angle in the elevational direction, the 3D-UBP reconstruction process added a weight to the back projected photoacoustic signals at different elevational divergence angles. To accurately reconstruct objects in the Fraunhofer zone, the photoacoustic signals were back-projected from virtual transducers located at the transition points between the Fresnel and Fraunhofer zones. A virtual point detector with applications to photoacoustic tomography is described in Yang, X., Li, ML., Wang, V. L., “Ring-based ultrasonic virtual point detector with applications to photoacoustic tomography,”Appl. Phys. Lett. 90, 251103 (2007), which is hereby incorporated by reference in its entirety. Sharing the same in-plane resolution as the 2D mode, the 3D-UBP process provided an improved elevational resolution of 5.6 mm.

The full-ring transducer array with 512 elements could spatially well sample objects—according to the spatial Nyquist criterion—within a field of view (FOV) of about 39 mm in diameter. To eliminate aliasing caused by under-sampling in regions outside of this FOV, the photoacoustic signals were low-pass filtered with cut-off frequencies determined by the distance to the center of the ring array.

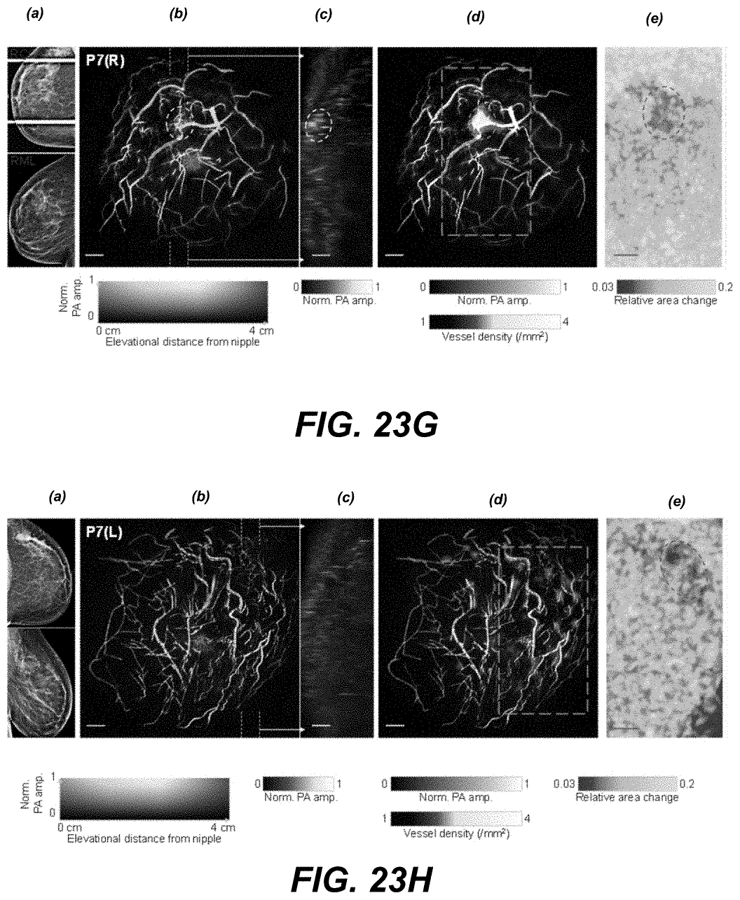

Using an implementation of a PACT method, each volumetric image was reconstructed with a voxel size of 1 mm in the elevational direction and 0.1×0.1 mm² on the horizontal plane. The reconstructed images were batch-processed all to improve contrast. In each horizontal slice, a Hessian-based Frangi vesselness filtration process was used to enhance the contrast of blood vessels with diameters ranging from 3 to 12 pixels. In each filtered slice, adaptive thresholding was used to segment blood vessels, followed by morphology filtration for single-pixel elimination. In the elevational direction of each filtered volumetric image, voxels were selected with the largest PA amplitudes and then projected their depths to form a 2D image. A median filtration was applied with a window size of 3×3 pixels to the depth image. Another median filtration with a window size of 6×6 pixels was further applied inside the segmented vessels to the segmented vessels' depths. Different RGB (red, green, blue) color values were assigned to discrete depths. Finally, the 2D depth-resolved color-encoded image was multiplied by the MAP image pixel by pixel to represent the maximum amplitudes. To further reduce noise and improve image quality, the above parameters in 2D slices were tuned at different depths, which resulted in the custom processing images in FIGS. 25A-H. As shown in these figures, the structures in all three sets of images match well with each other, showing the fidelity of the vesselness filtering and custom processing.

### B. Vascular Diameter Measurement

A PACT method of one implementation was used to measure vascular diameters by identifying vessel boundaries through a correlation-based template matching process. The templates were generated through simulation. The impulse responses of all ultrasonic transducers were used to simulate the images of vessels with different sizes (0.5-2.0 mm) and orientations. The diameters of vessels chosen from the PACT breast images were quantified by matching the reconstructed vessel images with the generated templates.

To separate fluctuations caused only by heart beats, frames with strong motion caused by body movement were first removed. The entire imaging field was then divided into 16 slightly overlapping subdomains. In each subdomain, the first frame was selected as the reference frame. The other frames were registered to it through rigid transformation, optimizing the frame-frame correlation. In each subdomain, a Gaussian filter with a radius of 0.2 mm was applied to all registered frames to reduce high spatial-frequency noise. A Fourier transformation was applied to each pixel's value through all the frames. The fluctuations in pixel values induced by arterial pulse propagation were quantified within the frequency range (1.0-1.6 Hz) of heartbeat cycles. The frequency range (1.0-1.6 Hz) of heartbeat cycles can be found in Bender, L., “Human Body” Crescent Books, New York, (1992).

### C. Tumor Segmentation

A PACT method, of one implementation, was used to identify breast masses by revealing a greater density of blood vessels, presumably due to angiogenesis, in tumor regions. To segment tumors automatically, the vessel skeleton was extracted and a vessel density (number of vessels/area) map of the breast was produced. The regions with the highest vessel density highlighted the breast mass of interest. The dense vessels in the nipple would affect the automatic tumor segmentation. Therefore, the shallowest slices containing the nipple were first removed. The remaining slices were used to generate the MAP image. A vessel mask was generated from the MAP by Hessian filtering and threshold-based segmentation. Based on the mask, vessel centerlines were extracted by removing boundary pixels. The vessel centerlines were broken into independent vessels at junction points.

To further reduce noise, the independent vessels with lengths less than 3 pixels (255-μm spatial resolution divided by 100-μm pixel size is approximately 3) were removed. A 2 mm×2 mm window was then used to scan the entire image. At each scanning location, the number of vessels (independent segments) inside the window was counted and assigned to the center pixel in the window. The vessel density was quantified as the number of vessels divided by the window area.

To demarcate breast tumors from MAP images, suspicious regions were first identified where blood vessel densities were higher than a threshold, which was set to each whole-breast's average plus 2.0 times the standard deviation. The number of pixels was counted in each contiguous region and the regions with pixel counts fewer than 1855 (18.55 mm²) were rejected to eliminate false positive cases. The remaining contiguous regions were labeled as tumors.

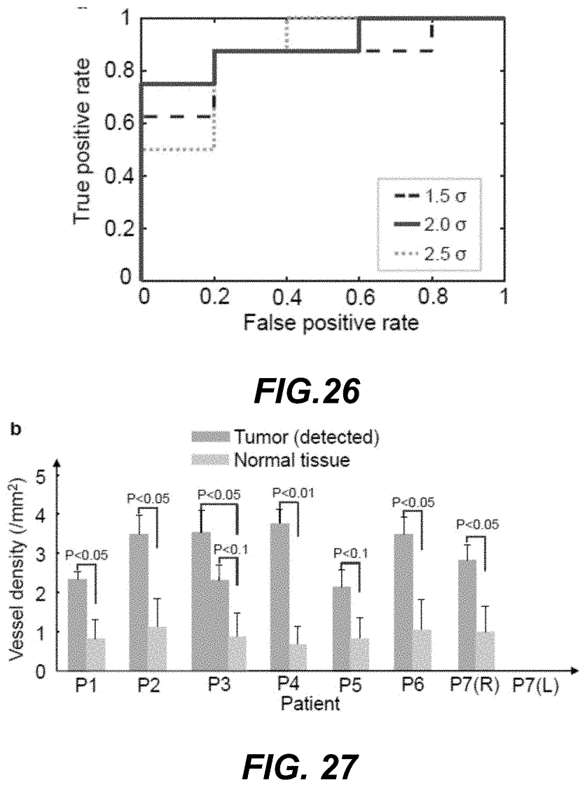

FIG. 30 is a plot of the receiver operating characteristic (ROC) curve of tumor identification based on the sizes of the contiguous high vessel density regions, according to an implementation. A threshold of numbers of pixels within (1855, 6379) produced a sensitivity of 89% and a specificity of 100%.

### D. Elastography Study

In a PACT method according to one implementation, the high imaging speed enabled differentiation in compliance between tumors and surrounding normal breast tissue, providing another contrast for detecting breast cancer. First, elastographic measurements were performed on a breast phantom as a test case. The phantom comprised a ball with 7% agar (mimicking breast tumor) embedded in a base of 2% agar (mimicking normal breast tissue). A discussion of breast tissue stiffness is described in Wellman, P. S., Howe, R. D., Dalton, E. & Kern, K. A., “Breast tissue stiffness in compression is correlated to histological diagnosis,” Technical Report. Harvard BioRobotics Laboratory, 1-15 (1999), which is hereby incorporated by reference in its entirety. Chopped human hair was uniformly distributed in the phantom to mimic small blood vessels. Working in 2D imaging mode, the PACT method quantified the relative area changes in a cross section when minor deformations were induced by periodic compressions (about 0.25 Hz) on top of the phantom. Due to the low elevational sectioning power of 2D imaging, objects in 2D frames were mainly influenced by coronal dilation instead of elevational displacement. Accordingly, the PACT elastography clearly revealed the agar ball with correct size and location as shown in FIGS. 25A and 25B. No obvious differences were observed in the concentration of the hair fiber between the balls and the phantom base.

To assess deformations over time, the first frame was taken as a reference. Other frames were registered to the first frame through a non-rigid demon algorithm in Matlab. An example of imaging matching can be found in Thirion, J P., “Image matching as a diffusion process: an analogy with Maxwell's demons,” Med. Image Anal. 2, 243-260 (1998), which is hereby incorporated by reference in its entirety. For each pixel of registered frames, the standard deviation (STD) of the value variations was calculated. Pixels with relatively small STDs were stably registered and were used for deformation quantification. The entire image was then segmented into 2 mm×2 mm squares. One stably registered pixel was chosen from each square, and triangular grids were further generated from these registered pixels. The triangular grids were mapped back to the original unregistered frames, and their areas were calculated. For each grid, Fourier transformation was applied to quantify the area variation at the frequency of periodic compression, and amplitudes were assigned to the pixels inside this triangle to generate the deformation map. To further reduce noise, 100 deformation maps were generated with randomly registered pixels in the squares. The final image is the average of the 100 deformation maps.

To conduct SHB-PACT elastography of the breast, patients were asked to breathe normally. The chest wall pushed the breast against the agar pillow, elevationally generating a deformation of the breast in the coronal plane. The same method was used to quantify the change of area between blood vessels in the breast. Tumors, being stiffer, could be identified in areas with less deformation than normal breast tissue.

### E. Images and Analysis Results

The PACT system described in Section IV(A) was used to identify eight of nine breast tumors by delineation of angiographic anatomy in the 3D image. These tumors were subsequently verified by ultrasound-guided biopsy. The automated tumor segmentation technique was used to highlight the tumors automatically. Tumors were clearly revealed by PACT techniques in all breasts even in radiographically dense breasts, which could not be readily imaged by mammography. Taking advantage of the high imaging speed, PACT techniques were implemented to take elastographic measurements of 2D images to detect tumors by assessing deformations caused by breathing. The elastography measurements identified the one tumor missed in angiographic imaging, and thus improved the sensitivity of tumor detection. At such high spatiotemporal resolutions, the PACT system was able to differentiate arteries from veins by detecting blood flow mediated arterial deformation at the heartbeat frequency.

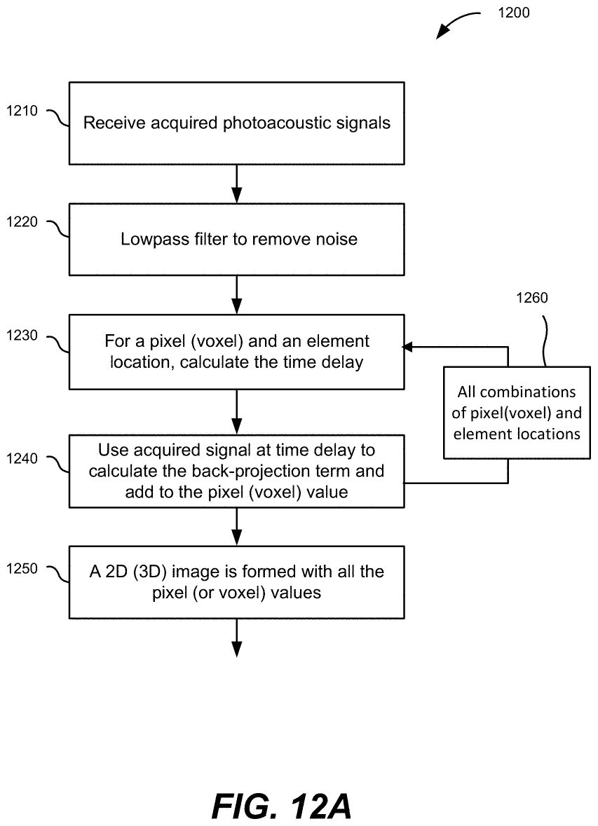

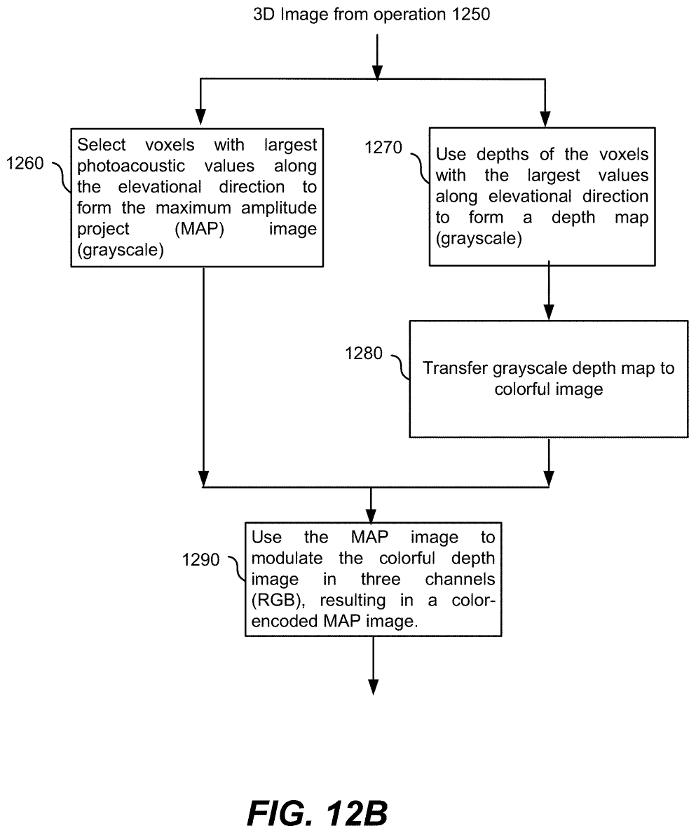

Before imaging breast cancer patients, the performance of the PACT system was assessed by imaging a 27-year-old healthy female volunteer. By scanning the transducer array elevationally through her right breast, within one breath hold (about 15 seconds), the angiographic anatomy was revealed from the nipple to the chest wall. FIGS. 13A-3D are PACT images of healthy breasts of a 27-year-old healthy female volunteer at four depths in increasing depth order from the nipple to the chest wall, according to an implementation. FIG. 13A is a PACT image at a depth of 0.5 cm from the nipple, according to an implementation. FIG. 13B is a PACT image at a depth of 1.5 cm from the nipple, according to an implementation. FIG. 13C is a PACT image at a depth of 2.5 cm from the nipple, according to an implementation. FIG. 13D is a PACT image at a depth of 4.0 cm from the nipple, according to an implementation. FIG. 14A is the same image from FIG. 13A with color-encoded depths shown in grayscale, according to an implementation. FIG. 14B is a close-up view of the region outlined in FIG. 14A with two vessels identified (V1 and V2). FIG. 14C is a graph of line spread plots of the two vessels identified in FIG. 14B. The color-encoded depth-resolved image shown in grayscale in FIG. 14A revealed the detailed angiographic structures of the entire breast, visualizing the vasculature down to an apparent vascular diameter of 258 μm as shown in FIG. 14B.

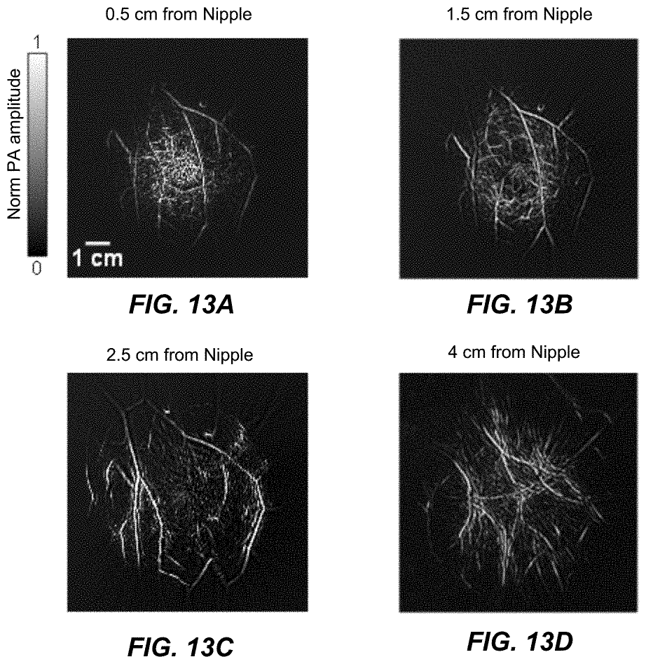

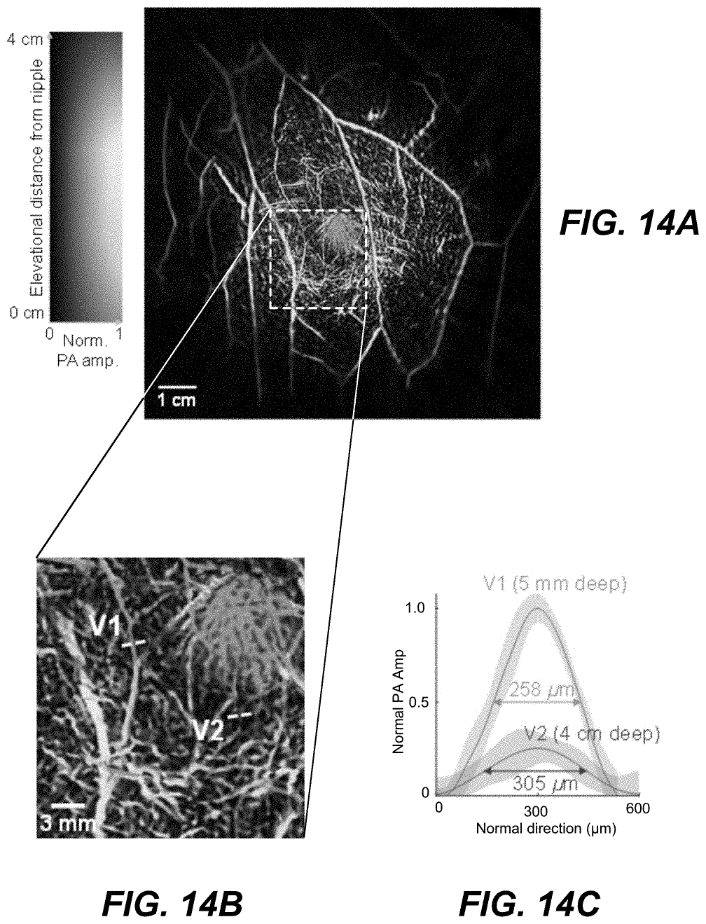

To measure the vascular diameters, vessel boundaries were identified in different slices through a correlation-based template matching technique such as, e.g., described in Tsai, P. S. et al., “Correlations of neuronal and microvascular densities in murine cortex revealed by direct counting and colocalization of nuclei and vessels,” J. Neurosci. 29, 14553-14570 (2009), which is hereby incorporated by reference in its entirety. FIGS. 15A-15C and FIGS. 16A-16C illustrate results from vascular diameter quantification, according to an implementation. FIG. 15A is an illustration with a numerically-simulated image of a cylinder with a diameter of 3 mm (left) and an experimental image of a rubber cylinder with a pre-known diameter of 3 mm, according to an implementation. FIG. 15B is a plot of photoacoustic amplitude distributions along the normal directions of the dashed lines in FIG. 15A of the numerically-simulated cylinder and the rubber cylinder. FIG. 15C is a plot of correlation coefficients between numerical cylinders with different diameters and the rubber cylinder, according to an implementation. FIG. 16A is an illustration with a numerically-simulated image of a cylinder with a diameter of 1.04 mm (left) and an in vivo image of a section of a human blood vessel, according to an implementation. FIG. 16B is a plot of photoacoustic amplitude distributions along the normal directions of the dashed lines in FIG. 16A of the numerically-simulated cylinder and the blood vessel. FIG. 16C is a plot of correlation coefficients between numerical cylinders with different diameters and the blood vessel, according to an implementation.

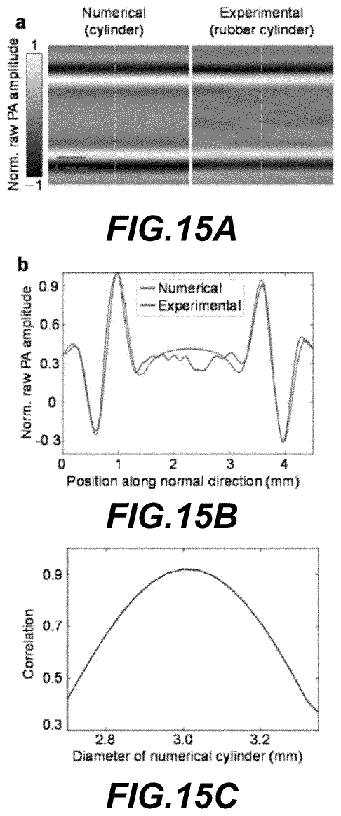

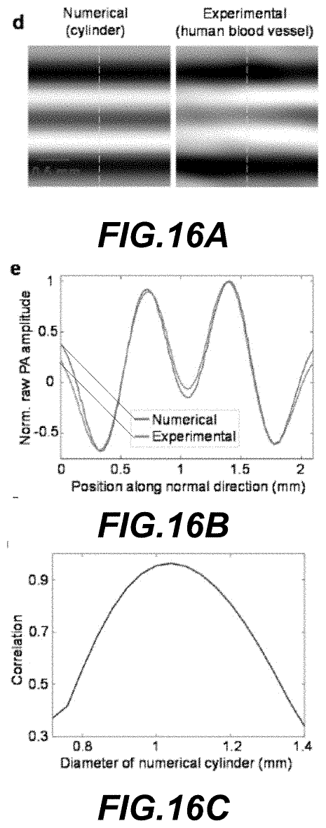

By measuring vascular diameters, the relationship between parent and daughter vessels at vascular bifurcations was further investigated, which is expressed by the junction exponent. Diameter relationships at vascular bifurcations is discussed in Witt, N. W. et al., “A novel measure to characterise optimality of diameter relationships at retinal vascular bifurcations,” Artery Res. 4, 75-80 (2010), which is hereby incorporated by reference in its entirety. A vessel tree was selected in the breast and marked five branch levels with distinct colors. FIG. 17 is a PACT image of a healthy breast with the selected vessel tree in the breast with the five vessel bifurcations, labeled from B1 to B5, according to an implementation. At each bifurcation, the diameter relationships between the parent vessel (Dₚₐᵣₑₙₜ) and daughter vessels (D_{daughter}) are presented on the right. X_{B} is the junction exponent, and R_{B} is defined as R_{B}=Dₚₐᵣₑₙₜ³/(D_{daughter_a}³+D_{daughter_b}³). At five vascular bifurcations (B1 to B5), the junction exponents were calculated as well as the ratios between the cube of the diameter of the parent vessel and the sum of the cubes of the diameters of the daughter vessels. For the eight subjects (one healthy volunteer and seven breast cancer patients), five vascular bifurcations were picked in each of their breasts and the average junction exponent quantified. The average junction exponent has a mean value of 2.63±0.34. FIG. 18 is a plot of the average junction exponents of the eight subjects, according to an implementation. The subjects included the healthy volunteer and patients. The subjects' ages ranged from 27 to 71. The junction exponents generally decreases with increasing age as discussed in Stanton, A. V. et al., “Vascular network changes in the retina with age and hypertension,” J. Hypertens 13, 1724-1728 (1995) and Witt, N. W. et al., “A novel measure to characterise optimality of diameter relationships at retinal vascular bifurcations,” Artery Res. 4, 75-80 (2010), which are hereby incorporated by reference in their entireties.

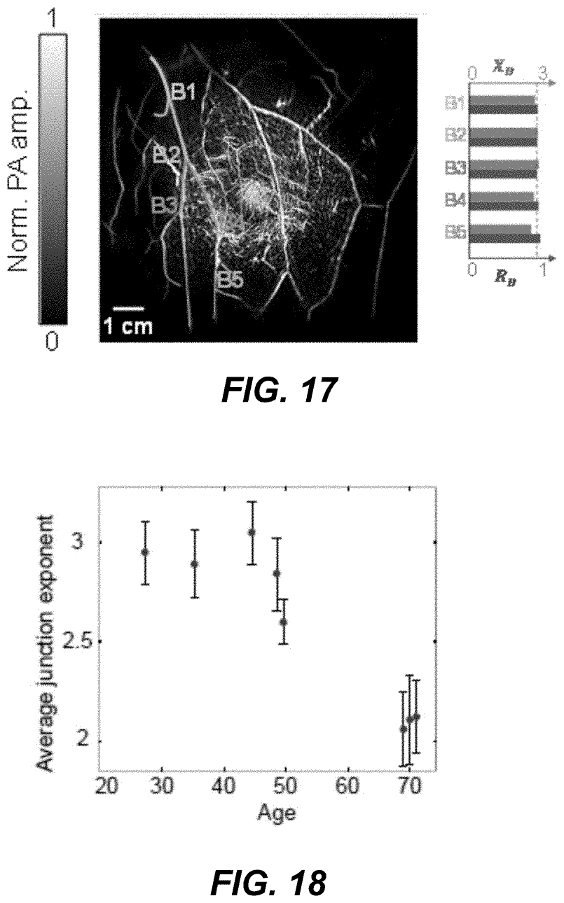

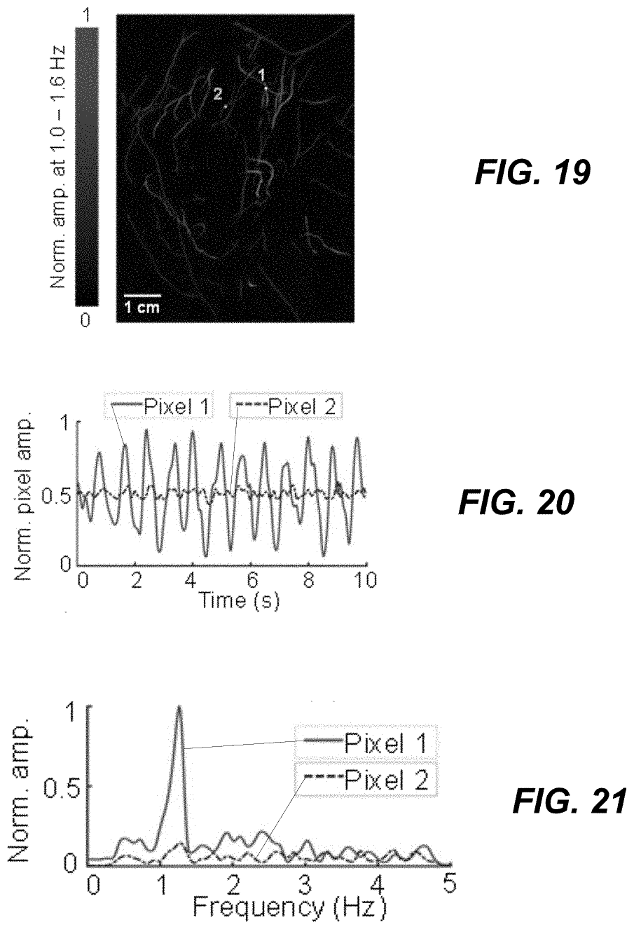

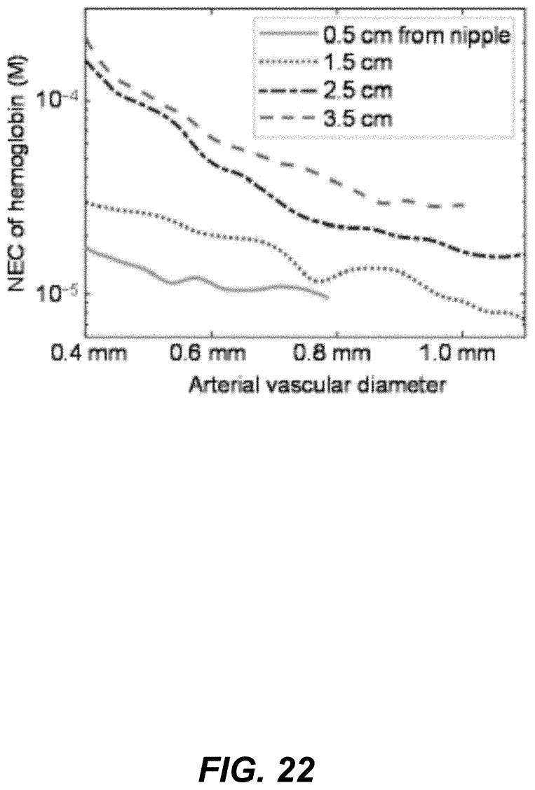

During a breath hold within 10 seconds, a cross section of the contralateral healthy breast in one of the breast cancer patients was imaged with the PACT system. Working in 2D mode at a 10 Hz frame rate, the PACT system continuously monitored arterial pulsatile deformation inside the breast by fixing the transducer array at a specific elevational position. An example of mechanotransduction can be found in Davies, P. F., “Flow-mediated endothelial mechanotransduction,”Physiol. Rev. 75, 519-560 (1995), which is hereby incorporated by reference in its entirety. The photoacoustic signals were analyzed pixel-wise in the frequency domain to identify arteries and veins according to the heartbeat frequency. Photoacoustic signals were analyzed pixel-wise in the frequency domain to identify arteries and veins according to the heartbeat frequency. FIG. 19 is a heartbeat-encoded arterial network mapping of a breast cross-sectional image of a healthy breast from a PACT system, according to an implementation. For illustration, a pixel was selected from one artery and one vein (highlighted by round dots 1 and 2 in FIG. 19). FIG. 20 is a plot of the pixel value fluctuation of the one artery and the one vein highlighted by dots in FIG. 19. FIG. 20 shows amplitude fluctuation in the time domain of the two pixels highlighted by the dots in FIG. 19. The pixel value in the artery shows changes associated with arterial pulse propagation. FIG. 21 is a plot in the Fourier domain of the pixel value fluctuations in FIG. 20. The periodic oscillation of the pixel values in the artery (arterial pixel values) indicates that the changes were the result of pulse waves propagating through the arterial network. The oscillation frequency further reveals the subject's heartbeat frequency of about 1.2 Hz as shown in FIG. 21. Considering that arterial blood has a relatively narrow range of oxygen saturation (sO₂), average photoacoustic signals from arteries can potentially be used to calibrate the local optical fluence (mJ/cm²) deep in the breast, and thus enable accurate quantification of functional parameters (e.g., blood sO₂) with an additional laser wavelength (e.g., 750 nm). Blood oxygen saturation is discussed in Yoshiya, I., Shimada, Y. & Tanaka, K., “Spectrophotometric monitoring of arterial oxygen saturation in the fingertip,”Med. Biol. Eng. Comput. 18, 27-32 (1980), Xia, J., “Calibration-free quantification of absolute oxygen saturation based on the dynamics of photoacoustic signals,” Opt. Lett. 38, 2800-2803 (2013), and Sivaramakrishnan, M. et al., “Limitations of quantitative photoacoustic measurements of blood oxygenation in small vessels,” Phys. Med. Biol. 52, 1349-1361 (2007), which are hereby incorporated by reference in their entireties.

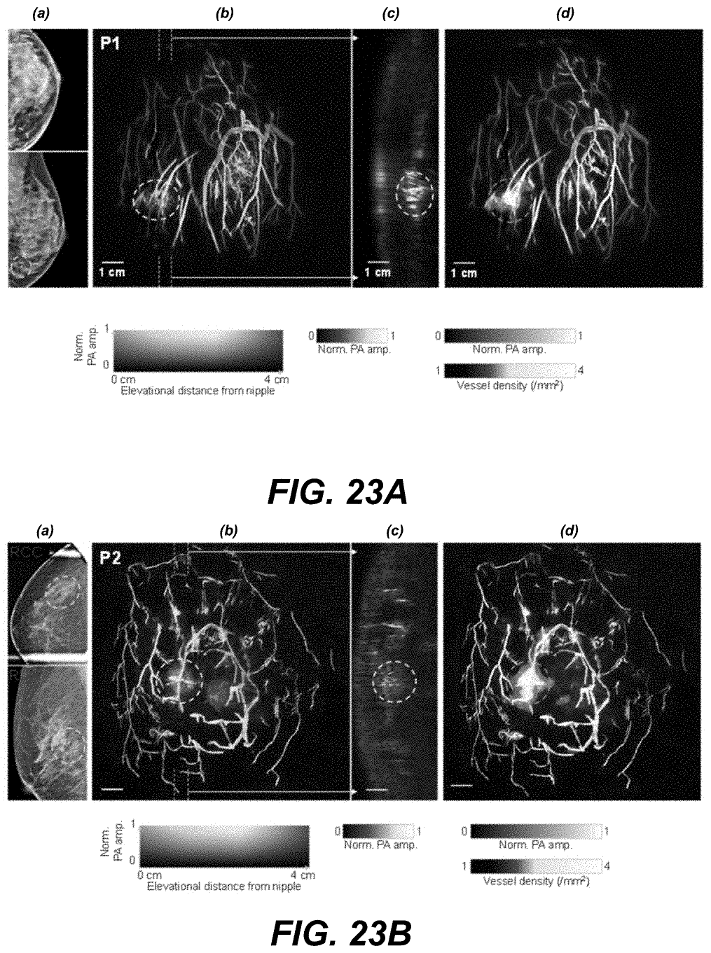

FIG. 22 is a plot of the noise-equivalent molar concentration (NEC) values plotted for arterial vessels with different diameters at different depths, according to an implementation. The breast size was C cup and the incident fluence of the PACT system was approximately 20 mJ/cm². This plot shows the noise-detection sensitivity of the PACT system, which enables the PACT system to detect breast tumors with fine details, making this imaging modality potentially useful for multiple applications in breast clinical care.

### 1. SBH-PACT of Breast Cancer Anatomy, Segmentation, and Elastography

The breasts of seven breast cancer patients, having breast sizes ranging from B cup to DD cup (over 99% of the U.S. population has breast sizes of DD cup or smaller according to “Average Breast Size” <<TheAverageBody.com>> (2015)) and skin pigmentations ranging from light to dark were imaged using the PACT system.

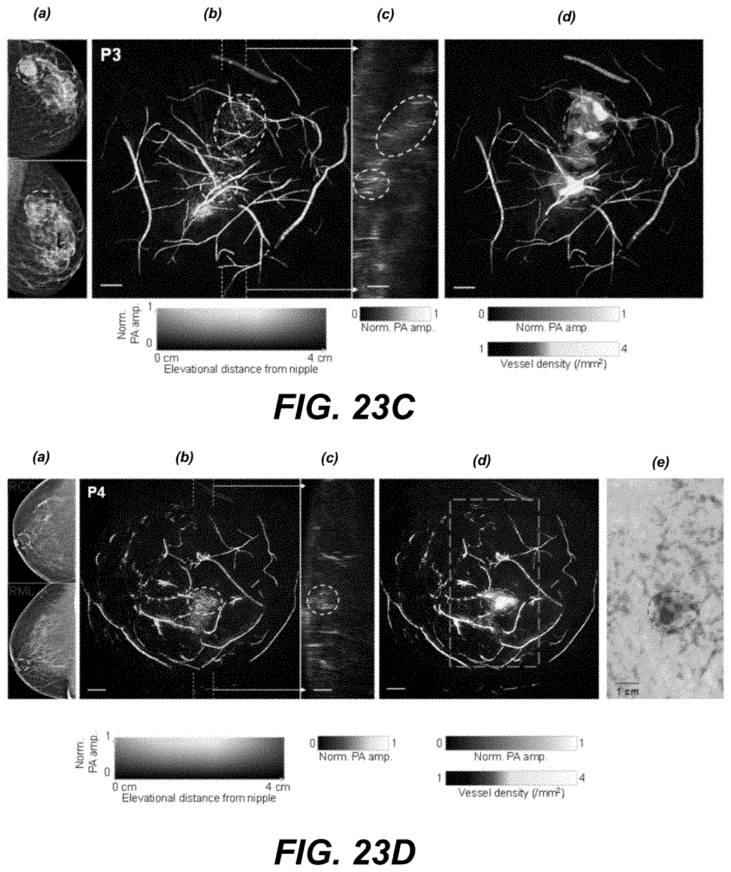

FIGS. 23A-H are images of breasts of the seven breast cancer patients. FIG. 23A are images of a breast of the first patient P1, according to an aspect. FIG. 23B are images of a breast of the second patient P2, according to an aspect. FIG. 23C are images of a breast of the third patient P3, according to an aspect. FIG. 23D are images of a breast of the fourth patient P4, according to an aspect. FIG. 23E are images of a breast of the fifth patient P5, according to an aspect. FIG. 23F are images of a breast of the sixth patient P6, according to an aspect. FIG. 23G are images of a right breast of the seventh patient P7, according to an aspect. FIG. 23H are images of a left breast of the seventh patient P7, according to an aspect. Patient P1 is a 48-year-old female patient with an invasive lobular carcinoma (grade 1/3). Patient P2 is a 70-year-old female patient with a ductal carcinoma in situ (microinvasion grade 3/3). Patient P3 is a 35-year-old female patient with two invasive ductal carcinomas (grade 3/3). Patient P 4 is a 71-year-old female patient with an invasive ductal carcinoma (grade 3/3). Patient P 5 is a 49-year-old woman with a stromal fibrosis or fibroadenoma. Patient P6 is a 69-year-old female patient with an invasive ductal carcinoma (grade 2/3). Patient P7 is a 44-year-old female patient with a fibroadenoma in the right breast and an invasive ductal carcinoma (grade 2/3) in the left breast. In these figures, the images in column (a) are X-ray mammograms of the affected breasts where label LCC indicates left cranial-caudal, label LLM indicates left lateral-medio, label LML indicates left mediolateral, label LMLO indicates left mediolateral-oblique, label RCC indicates right cranial-caudal, and label RML indicates right medio-lateral. In these figures, the images in column (b) are depth-encoded angiograms of the eight affected breasts acquired by the PACT system. The breast tumors are identified by circles in Patients P2-P8. In these figures, the images in column (c) are maximum amplitude projection (MAP) images of thick slices in sagittal planes marked by white dashed lines in depth-encoded angiograms. In these figures, the images in column (d) are automatic tumor detection on vessel density maps using PACT techniques. The tumors are identified by circles. Background images in gray scale are the MAP of vessels deeper than the nipple. In these figures, the images in column (e) are maps of the relative area change during breathing in the regions outlined by dashed boxes in the angiographic images. The same tumors are identified by circles. The elastographic study using PACT techniques began with Patient 4, and revealed all imaged tumors, including the undetected one in FIG. 23H.

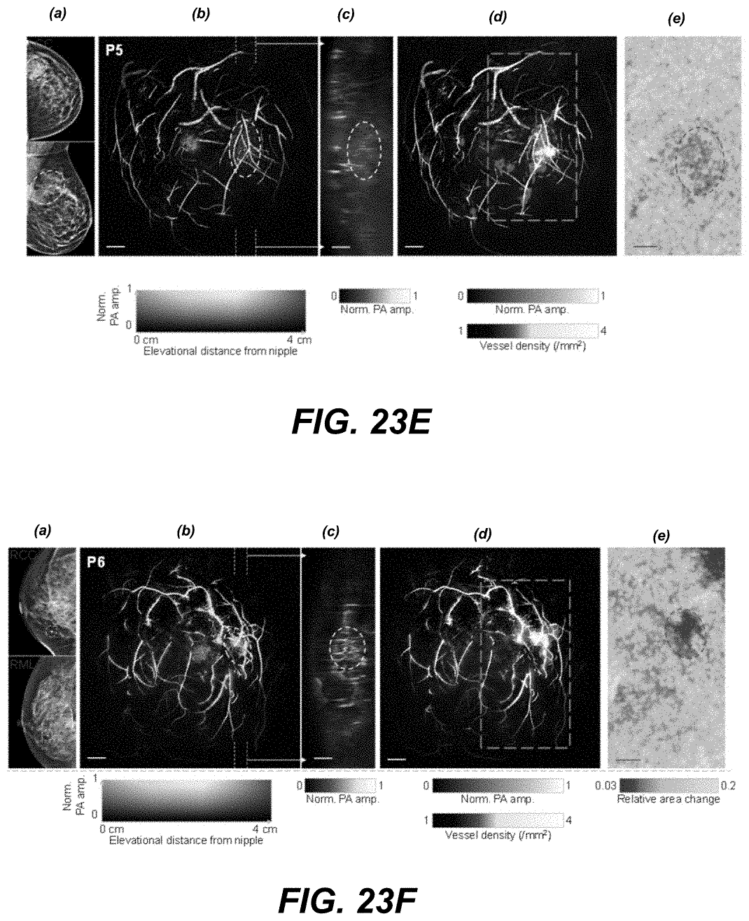

FIGS. 24A-H are side-by-side comparisons of PACT images of depth-encoded angiograms that were batch processed without vesselness filtering, batch processed with vesselness filtering, and custom processed, respectively. All the tumors that are enclosed by dashed circles can be visualized in the batch-processed images. The tumor in the left breast of patient P7 is invisible in the angiograms although it is visible in the photoacoustic elastogram shown in FIG. 23H. FIG. 24A are images of a breast of the first patient P1, according to an aspect. FIG. 24B are images of a breast of the second patient P2, according to an aspect. FIG. 24C are images of a breast of the third patient P3, according to an aspect. FIG. 24D are images of a breast of the fourth patient P4, according to an aspect. FIG. 24E are images of a breast of the fifth patient P5, according to an aspect. FIG. 24F are images of a breast of the sixth patient P6, according to an aspect. FIG. 24G are images of a right breast of the seventh patient P7, according to an aspect. FIG. 24H are images of a left breast of the seventh patient P7, according to an aspect.

Angiogenesis, which plays a central role in breast cancer development, invasion, and metastasis, is the essential hallmark by which PACT techniques may be able to differentiate lesions from normal breast tissue. Well correlated with the tumor locations shown in mammograms and reported by ultrasound-guided biopsy, the PACT images in FIGS. 23A-H and FIGS. 24A-H were used to determine eight of the nine tumors by observing higher blood vessel densities associated with tumors in the depth-encoded images. Tumor-containing slices were selected perpendicular to the chest wall (marked by dashed lines in column (b) of FIGS. 23A-H). In these sagittal (side-view) images, the same tumors, where higher PA amplitude is shown, can be seen at corresponding locations in column (c) of FIGS. 23A-H). In the X-ray mammograms of Patient 1 (P1) and Patient 6 (P6), the lesions in the dense breasts are barely distinguishable. In comparison, the PACT images clearly revealed the tumors not readily seen in mammograms, notwithstanding the high radiographical density of the breast.

The PACT method with tumor segmentation may be used to distinguish tumors automatically, which may be beneficial in a clinical setting. Presumably due to angiogenesis, tumors appear as regions of denser blood vessels in PACT images. When implementing the PACT method to segment tumors automatically, the vessel skeleton was extracted and a vessel density map was produced of the breast (local vessel number/local area). The regions with the highest vessel density highlight the breast tumors as shown in column (d) of FIGS. 23A-H.

In addition to direct observation of blood vessel density, the PACT system detected the difference in compliance between tumors and surrounding normal breast tissue, providing an alternate concurrent contrast to detect breast cancer. Before performing elastography on breast cancer patients, this PACT method was used to image breast-mimicking phantoms. FIG. 25A is a PACT image of a cross-sectional image of the phantom acquired by the PACT system, according to an implementation. Hundreds of chopped human hairs were uniformly distributed in the phantom to mimic small blood vessels. To mark the location for comparison, two crossed tungsten wires (indicated by yellow arrows) were placed inside the ball (enclosed by the red dashed circle), which had a higher agar concentration to mimic a breast tumor. FIG. 25B is a PACT elastographic image of the cross-section in FIG. 25A. Identified by the dashed circle, the location of the agar ball is revealed correctly. Working in 2D imaging mode, the PACT system quantified the relative area changes in a breast cross section when minor deformations were caused by breathing. Because breast tumors are generally less compliant than normal breast tissue, the regions with lower relative area changes indicated the breast tumor in column (e) images in FIGS. 23D-23H. A discussion of breast tumors being less compliant than normal breast tissue is discussed in Fenner, J. et al. Macroscopic stiffness of breast tumors predicts metastasis. Sci. Rep. 4, 5512 (2014), which is hereby incorporated by reference in its entirety. Unlike ultrasonic elastography, The PACT elastography utilized the contrast of hemoglobin and formed area-quantificational grids between vessels. From only angiographic anatomy detailed by the PACT method, the only tumor missed was located in a marginal region of a D cup breast (P7(L)), where light illumination was insufficient. However, with the addition of PACT elastography, the missed tumor was identified. Taking advantage of the short time requirement for elastographic measurement (about 10 seconds), the PACT techniques can observe both blood vessel density and tissue compliance simultaneously within about 30 seconds. Taken together, these two PACT measurements may be able to improve the sensitivity of breast cancer detection.

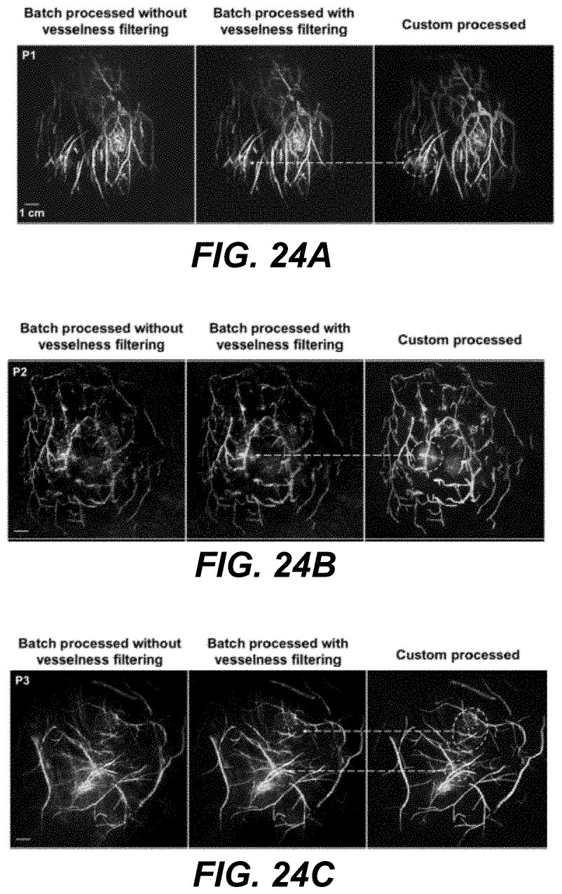

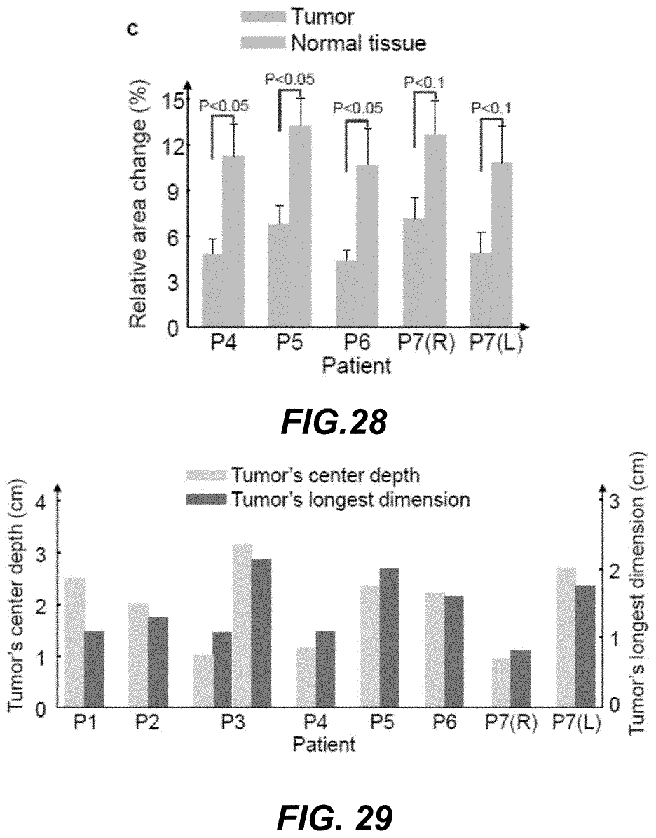

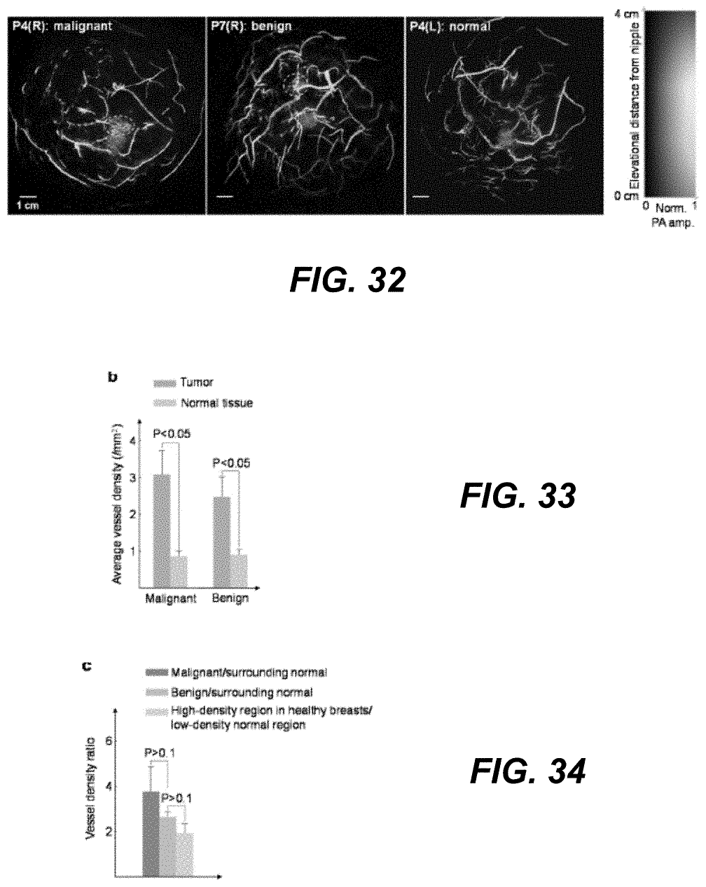

During an evaluation, a PACT system was used to identify eight of the nine biopsy-verified tumors by assessing blood vessel density. Moreover, the initially undetected tumor was subsequently revealed by elastographic SBH-PACT. Pathology reports showed two benign tumors (Patient 5, stromal fibrosis or fibroadenoma; Patient 7, right, fibroadenoma), one ductal carcinoma in situ (DCIS) with a 3/3 nuclear grade (Patient 2), and six invasive carcinomas (all other cases). Angiogenesis serves as a basis for tumor identification. Considering the diversity among the subjects, high blood vessel densities were defined as values greater than the whole-breast average plus (a) 1.5, (b) 2.0, or (c) 2.5 times the standard deviation, respectively. The ratios of average vessel density were calculated and compared between the high-density region and the normal density region in each affected and contralateral breasts shown in FIGS. 31A-31G. Receiver operating characteristic (ROC) curves were plotted by varying the threshold of the ratios from 1 to 6. FIG. 26 is a plot of the receiver operating characteristic (ROC) curves of breast tumor detection based on blood vessel density, according to an aspect. Based on the data from the finite set of subjects, option (b) yielded the largest area (0.90) under the ROC curve. A threshold within (2.26, 2.58) produced a sensitivity (true positive rate) of 88% and a specificity (true negative rate) of 80%. Training and testing studies were performed by obtaining a threshold based on randomly picked six breasts (training set) and then applying the threshold to the remaining seven breasts (testing set). This procedure was repeated ten times and the average sensitivity and specificity calculated. FIG. 35 is a table of sensitivities and specificities of tumor detection based on vessel-density thresholds obtained from the training data sets, according to an implementation.

FIGS. 31A-H are side-by-side comparisons between the left and right breast PACT images of each patient, according to an implementation. FIG. 31A are images of the left breast of the first patient P1, according to an aspect. FIG. 31B are images of the breasts of the second patient P2, according to an aspect. FIG. 31C are images of the breasts of the third patient P3, according to an aspect. FIG. 31D are images of the breasts of the fourth patient P4, according to an aspect. FIG. 31E are images of the breasts of the fifth patient P5, according to an aspect. FIG. 31F are images of the breasts of the sixth patient P6, according to an aspect. FIG. 31G are images of the breasts of the seventh patient P7, according to an aspect.

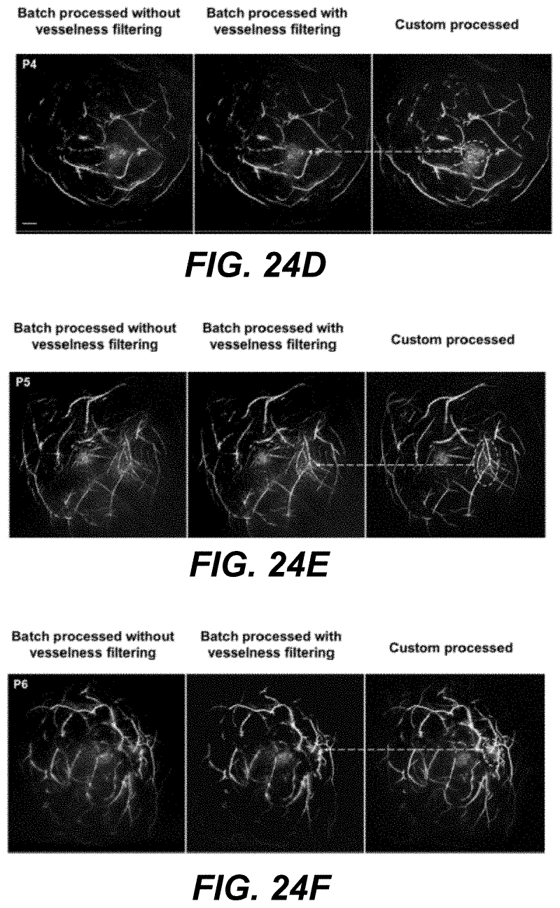

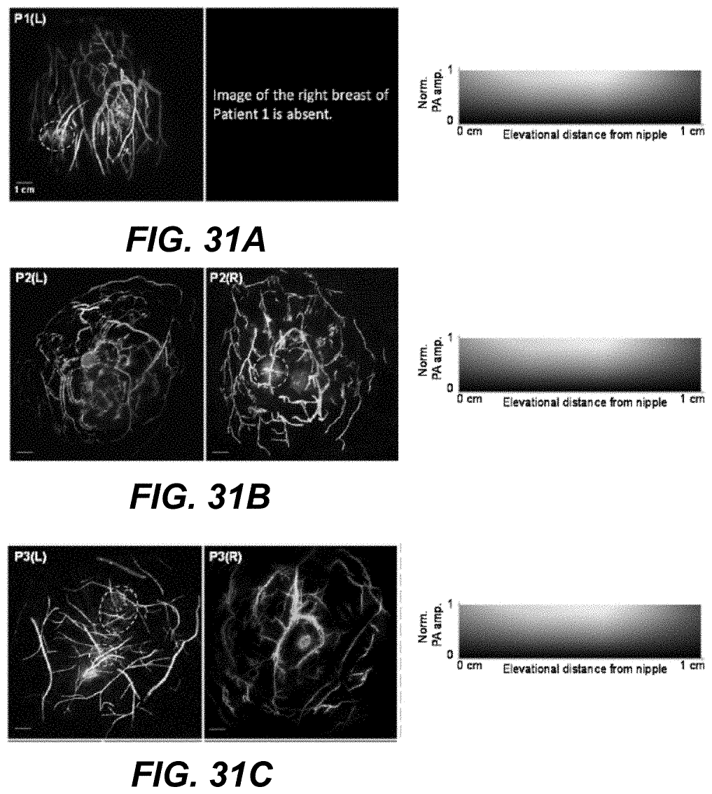

The tumors were then demarcated in each breast and the average vessel densities inside and outside the tumors were computed using one or more methods described in Section III. The average vessel density ratios between the tumors and the surrounding normal breast tissues were 3.4±0.99. FIG. 27 is a bar chart of the average vessel density in each tumor and the surrounding normal breast tissue, according to an aspect. In addition, the mean of the average vessel density ratios of the six malignant tumors was 1.4 times higher than that of the two benign ones. FIG. 33 is a plot of the average vessel densities of tumors and surrounding normal tissues, according to an implementation.

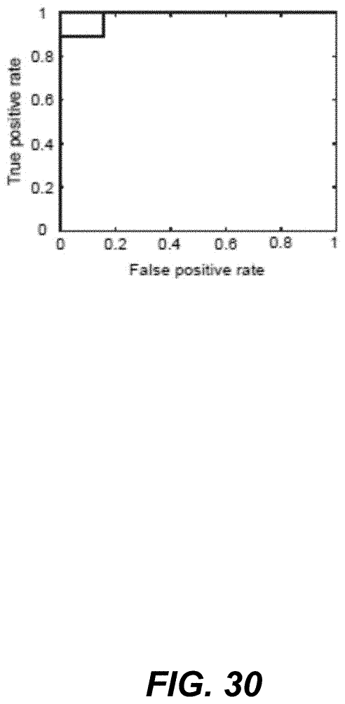

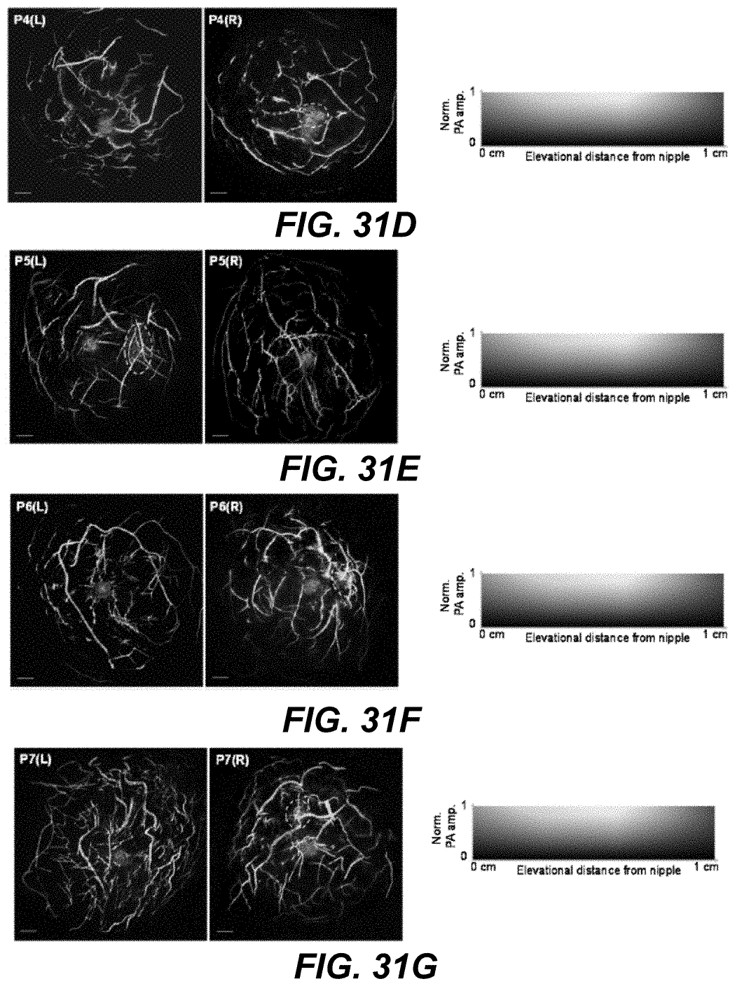

FIG. 32 is an illustration of three PACT images of breasts, according to an implementation. The first PACT image of the right breast of patient 4, P4(R), has a malignant tumor P4(R). The second PACT image of the right breast of patient 7, P7(R) has a benign tumor. The third PACT image of the left breast of patient 4, P4(L) does not have a tumor. FIG. 33 is a plot of the average vessel densities of tumors and surrounding normal tissues, according to an implementation. FIG. 34 is a plot of the average vessel density ratio, according to an implementation. The average vessel density ratio between the tumor and normal tissue of malignant tumors is approximately 1.4 times higher than that of benign ones.

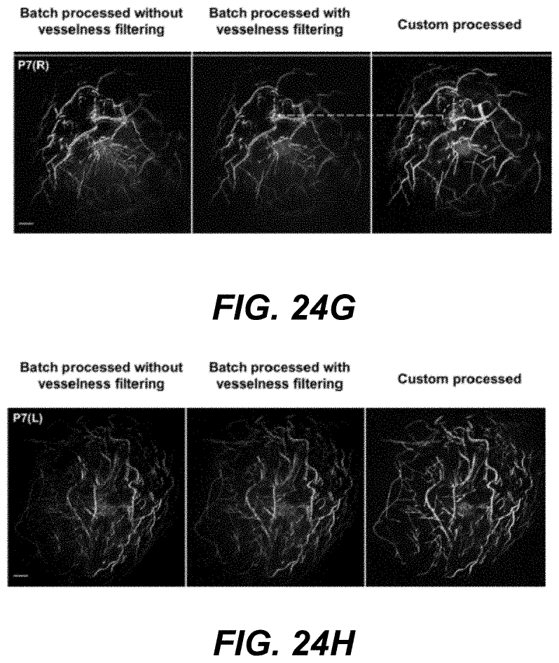

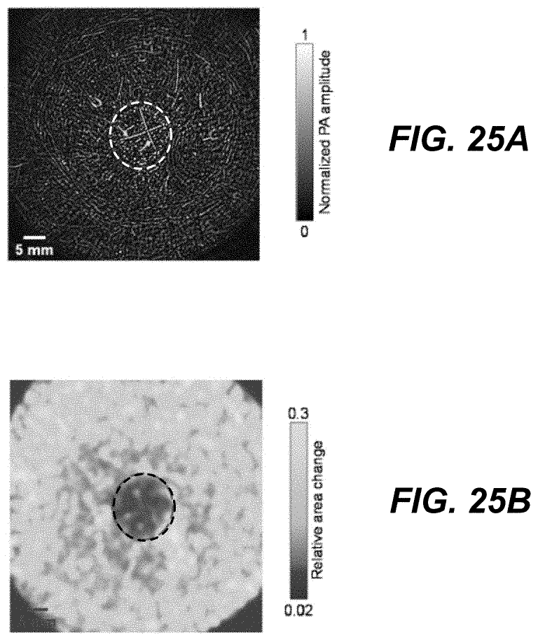

Since the elastography study began with Patient 4, PACT elastography identified all five tumors in the subsequent four patients. FIG. 28 is a bar chart of the relative area change in each tumor and the surrounding normal breast tissue caused by breathing, according to an aspect. The elastography study was started with patient 4, P4. The average breath-induced area change in tumors was around 2 times lower than that in normal breast tissue. As the patient recruitment protocol excluded patients with a mass smaller than 1 cm in diameter in this study, the longest dimension of the smallest tumor detected was approximately 0.8 cm. FIG. 29 is a bar chart of the longest dimension and center depth of each tumor, according to an aspect. This tumor was located in the right breast of Patient 7, who was recruited due to a larger tumor in her left breast. However, with 255-μm spatial resolution and refined noise-equivalent sensitivity, PACT techniques have the potential to detect smaller breast cancers once angiogenesis sufficiently progressed. Patient 3 had DD cup breasts, and her breast was compressed against the chest wall to roughly a cylinder. The tumor in her breast had a depth of ˜3.2 cm (elevational distance from the nipple), which was the deepest among the recruited patients. In certain cases, a PACT system integrates deep penetration, high spatiotemporal resolution, sensitive breast cancer detection, and 2D/ 3D switchable modes. One-to-one mapped low-noise amplifiers and DAQ circuits enabled 2D imaging using a single laser impulse or 3 imaging of an entire breast within a single breath hold (less than about 15 seconds or less than about 10 seconds). The high imaging speed avoided respiration-induced motion artifacts and enabled detection of breast tumors by detailing tumor associated angiogenesis. The donut-shaped optical illumination and panoramic acoustic detection provided a more uniform fluence distribution in deep tissue and best in-plane coverage of ultrasound reception, respectively, delivering high image quality. Furthermore, considering the low cancer detection rate (0.41%), even though modern mammography uses a low dose of ionizing radiation, the risk-to-benefit ratio (e.g., 8%-17% for 40-50 year-old women) is considered high. The risk-to-benefit ratio is discussed in Hendrick, R. E. & Tredennick, T., “Benefit to radiation risk of breast-specific gamma imaging compared with mammography in screening asymptomatic women with dense breasts,” Radiology 281, pp. 583-588 (2016) and Jung, H., “Assessment of usefulness and risk of mammography screening with exclusive attention to radiation risk,” Radiologe 41, 385-395 ( 001), which are hereby incorporated by reference in their entireties. In comparison, SBH-PACT requires neither ionizing radiation nor an exogenous contrast agent, yielding zero risk. The cancer detection rate is discussed in Cancer rate (per 1,000 examinations) and Cancer Detection Rate (per 1,000 examinations) for 1,838,372 Screening Mammography Examinations from 2004 to 2008 by Age—based on BCSC data through 2009. NCI-funded Breast Cancer Surveillance Consortium (HHSN261201100031C).

In certain cases, the laser beam was broadened into a donut shape with an outer diameter of about 10 cm, depositing light with an average laser fluence of about 20 mJ/cm2 on the breast surface (which is about ⅕ of the American National Standards Institutes safety limit). This outer radius covered most breasts and provided satisfactory SNR in breast images. Merely assessing blood vessel density, one tumor located in an insufficiently illuminated marginal region of a D cup breast was not detected (P7(L) in column (a) of FIG. 24H). Another implementation of the PACT system can improve sensitivity in breast cancer detection if equipped with a more energetic laser, which would enlarge the illumination area and increase the optical fluence.

In certain aspects, a PACT method includes an automatic tumor segmentation algorithm that may make it easier to recognize tumors by highlighting the suspicious affected region with the highest vessel density. In addition, the high 2D imaging speed of PACT techniques (e.g., 10 Hz frame rate) enabled the performance of elastographic measurements that may help improve breast cancer detection. The capability of PACT techniques to map arterial distribution can potentially be useful in diagnosing artery-related diseases. Discussions related to artery-related diseases can be found in Caplan, L. R., “Carotid-artery disease,” N. Engl. J. Med. 315, 886-888 ( 196), Libby, P., Ridker, P. M. & Maseri, A., “Inflammation and atherosclerosis,” Circulation 105, 1135-1143, (2002), and Ouriel, K., “Peripheral arterial disease,” Lancet 358, 1257-1264 (2005), which are hereby incorporated by reference in their entireties. In addition, the knowledge of vessel diameters and average PA signals from arteries can be used to calibrate the local optical fluence, thus providing accurate spectral sO₂ measurement in deep tissue.

The PACT techniques may provide a tool for future clinical use including not only screening, but also diagnostic studies to determine extent of disease, to assist in surgical treatment planning, and to assess responses to neoadjuvant chemotherapy. Compared to mammography, PACT techniques utilize non-ionizing radiation, show promise for sensitivity in radiographically dense breasts, and impose less or no pain by only slightly compressing the breast against the chest wall. Because the average hemoglobin concentration in malignant tumors is generally twice that in benign tumors, PACT techniques may have the potential to distinguish malignant tumors from benign tumors by quantifying blood vessel densities in the tumor. For example, one implementation of a PACT system was used to compare malignant and benign tumors by comparing vessel density ratio. The results are shown in FIG. 34. Based on the vessel density in the two benign tumors and the six detected malignant ones of this number of patients, the threshold of the vessel density ratio between tumors (either malignant or benign) and healthy tissues may be set within the range of (2.72, 2.76) to differentiate malignant tumors from benign ones. Using hemoglobin as the contrast, PACT techniques may potentially monitor breast cancer's response to neoadjuvant chemotherapy by acquiring information similar to that of contrast-enhanced MRI, yet with finer spatial resolution, higher imaging speed, and only endogenous contrast. A discussion of comparisons of benign and malignant tumors in other imaging techniques can be found in Ntziachristos, V., Yodh, A., Schnall, M. D. & Britton, C., “MRI-guided diffuse optical spectroscopy of malignant and benign breast lesions,” Neoplasia 4, 347-354 (2002), Zhu, Q. et al., “Benign versus malignant breast masses: optical differentiation with US-guided optical imaging reconstruction,” Radiology 237, 57-66 (2005), and Choe, R. et al., “Differentiation of benign and malignant breast tumors by in-vivo three-dimensional parallel-plate diffuse optical tomography,” J. Biomed. Opt. 14, 024020 (2009), which are hereby incorporated by reference in their entireties.

### 2. Standard of Care Work-Up, Percutaneous Biopsy, and Pathologic Diagnosis

The PACT imaging in this section was performed after a standard of care (SOC) work-up, but in advance of percutaneous biopsy. This order of events was designed to minimize confounding imaging findings related to biopsy-induced hemorrhage. Patients underwent only one PACT imaging study, which took less than 10 minutes. Both the contralateral and affected breasts were imaged. For the abnormal breast, the tumor size, tumor depth, blood vessel density, and signal amplitude in the breast images were analyzed. The analysis of tumor size/depth was further compared with the standard imaging results (mammography and ultrasonography). To identify the tumor types and grades, histopathology results from the SOC biopsy were used as the ground truth for interpretation of the results.

Using established clinical protocols, abnormalities were identified either through routine screening mammography, or diagnostic evaluation in symptomatic patients. Pre-biopsy work up included combinations of digital mammography, digital breast tomosynthesis, and ultrasound. Formal BI-RADS (breast imaging, reporting and data system) assessments were assigned in all cases, with appropriate recommendation for biopsy. Image-guided percutaneous biopsy was obtained using real-time ultrasound guidance and a 12-guage or 14-guage spring-loaded biopsy needle (chosen at the discretion of the performing physician.). Core specimens were submitted in formalin to the pathology department for histologic analysis as per normal routine at the institution. All cases were reviewed following receipt of the final pathology report to determine radiologic-pathologic correlation. Some patients underwent contrast enhanced breast MRI following confirmation of malignancy.

Modifications, additions, or omissions may be made to any of the above-described embodiments without departing from the scope of the disclosure. Any of the embodiments described above may include more, fewer, or other features without departing from the scope of the disclosure. Additionally, the steps of described features may be performed in any suitable order without departing from the scope of the disclosure. Also, one or more features from any embodiment may be combined with one or more features of any other embodiment without departing from the scope of the disclosure. The components of any embodiment may be integrated or separated according to particular needs without departing from the scope of the disclosure. For example, it would be understood that while certain PACT systems are described herein with a linear stage, another mechanism may be used.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic medium such as a hard-drive or a floppy disk, or an optical medium such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed embodiments have been described in some detail to facilitate understanding, the described embodiments are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

1. A photoacoustic computed tomography (PACT) system, comprising:
at least one pulsed or modulated light source;
an ultrasonic transducer array comprising unfocused transducer elements, each unfocused transducer element having a field-of-view in a range of 5 degrees to 30 degrees in a direction along an axis; and
a scanning mechanism configured to move and/or scan the ultrasonic transducer array along the axis.

2. The PACT system of claim 1, wherein the ultrasonic transducer array is a full-ring ultrasonic transducer array and the unfocused transducer elements are distributed around a circumference of a ring centered about the axis.

3. (canceled)

4. The PACT system of claim 1, wherein the scanning mechanism is configured to:
(1) move the ultrasonic transducer array to one or more locations along the axis and hold at each location for a first time period; and/or
(2) scan the ultrasonic transducer array between two locations along the axis during a second time period.

5. (canceled)

6. The PACT system of claim 1, further comprising an axicon lens followed by an engineered diffuser configured to convert a light beam from the at least one pulsed or modulated light source to a donut-shaped illumination beam or to a uniform circular illumination beam.

7. (canceled)

8. The PACT system of claim 1, further comprising a plurality of preamplifiers and a plurality of data acquisition systems in one-to-one mapped association with the plurality of unfocused transducer elements, the plurality of data acquisition systems configured to record the photoacoustic signals while the scanning mechanism moves/scans the ultrasonic transducer array along the axis.

9. The PACT system of claim 1, further comprising a computing system configured to execute instructions to reconstruct a plurality of 2D images and/or a 3D volumetric image using photoacoustic signals recorded while the scanning mechanism moves/scans the ultrasonic transducer array along the axis.

10. (canceled)

11. The PACT system of claim 9, wherein the computing system is further configured to execute instructions to determine an elastogram using the plurality of 2D images and/or calculate a vessel density map using the 3D volumetric image.

12. The PACT system of claim 11, wherein the computing system is further configured to execute instructions to identify one or more regions with a potential mass using the elastogram and/or the vessel density map.

13. (canceled)

14. The PACT system of claim 9, wherein the computing system is further configured to execute instructions to perform an automated tumor segmentation process to identify one or more regions with a potential mass using the 3D volumetric image.

15-18. (canceled)

19. The PACT system of claim 1, wherein the PACT system is configured to be switchable between:
(i) a 2D mode, wherein in the 2D mode the ultrasonic transducer array is moved to one or more locations along the axis and hold at each location for a first time period; and
(ii) a 3D mode, wherein in the 3D mode the ultrasonic transducer array is scanned between two locations along the axis during a second time period.

20. A photoacoustic computed tomography (PACT) method, the method comprising:
causing at least one pulsed light source to generate one or more light pulses configured to illuminate a specimen being imaged;
controlling a scanning mechanism to move and/or scan the ultrasonic transducer array in a direction along an axis, wherein the ultrasonic transducer array includes a plurality of unfocused transducer elements, wherein the ultrasonic transducer array is moved/scanned in the direct along the axis while each of a plurality of unfocused transducer elements detects photoacoustic waves within a field-of-view in a range of 5 degrees to 30 degrees in the direction along the axis; and
reconstructing a plurality of 2D images and/or a 3D volumetric image using photoacoustic signals recorded while the scanning mechanism moves/scans the ultrasonic transducer array in the direction along the axis.

21. The PACT method of claim 20, wherein the plurality of 2D images are reconstructed from photoacoustic signals recorded while the ultrasonic transducer array is held at a location along the axis.

22. The PACT method of claim 20, further comprising causing the scanning mechanism to hold the ultrasonic transducer array at each of one or more locations along the axis for a first time period, wherein a 2D image is reconstructed for each location along the axis.

23. The PACT method of claim 20, further comprising generating an arterial vessel mapping of the specimen being imaged using the plurality of 2D images and/or measuring vascular diameters in the specimen being imaged using the 3D volumetric image.

24. (canceled)

25. The PACT method of claim 20, wherein the 3D volumetric image is reconstructed from photoacoustic signals recorded while the ultrasonic transducer array is scanned between two locations along the axis during a second time period.

26. (canceled)

27. The PACT method of claim 25, wherein the second time period is shorter than 10 seconds or shorter than 15 seconds.

28. The PACT method of claim 20, further comprising determining an elastogram using the plurality of 2D images and/or calculating a vessel density map using the 3D volumetric image.

29. The PACT method of claim 28, further comprising identifying one or more regions with a potential mass using the elastogram and/or the vessel density map.

30. The PACT method of claim 20, wherein the specimen is a human breast plurality of 2D images are acquired at a rate of at least 10 Hz.

31-33. (canceled)

34. The PACT method of claim 30, wherein the one or more one or more light pulses are converted into a donut beam configured to circumferentially illuminate the human breast.

35. The PACT method of claim 30,
wherein the ultrasonic transducer array is a full-ring ultrasonic transducer array with unfocused transducer elements distributed around a circumference of a ring centered about the axis,
wherein the circumference is at least 200 mm, and
wherein the full-ring ultrasonic transducer array is moved/scanned to an outside surface of the human breast while photoacoustic signals are recorded.

36-45. (canceled)
